(19) United States
(12) Patent Application Publication
(10) Pub. No.: US 2003/0167513 A1
(43) Pub. Date: Sep. 4, 2003

(54) Selection and use of isopropylmalate synthase (IPMS) mutants desensitized in L-leucine negative feedback control

(76) Inventors: George S. Mourad, Ft. Wayne, IN (US); Damian J. Junk, Tucson, AZ (US)

Correspondence Address: BARNES & THORNBURG, 2600 CHASE PLAZA, 10 SOUTH LASALLE STREET, CHICAGO, IL, 60603, US

(21) Appl. No.: 10/306,905

(22) Filed: Nov. 27, 2002

Related U.S. Application Data
(60) Provisional application No. 60/339,895, filed on Nov. 30, 2001.

(51) Int. Cl. A01H 1/00; C12N 15/82; C12P 13/04

(52) U.S. Cl. 800/278; 435/468; 435/106

(57) ABSTRACT

Methods and compositions to overproduce L-leucine in plants, thereby increasing the nutritional value of plants use mutant forms of plant IPMS genes. Isopropylmalate synthase (IPMS) genes of the leucine biosynthetic pathway in a higher plant have been identified and isolated. Methods to engineer mutant forms of IPMS based on the wild-type sequence information are disclosed. Full length cDNAs of three loci in Arabidopsis namely IMS1, IMS2, and IMS3 were analyzed and their expression patterns characterized. Mutant forms of IPMS genes of the present invention, in particular IMS2, are selected after mutagenesis, and transformed into plants. The plants transformed with a desired mutant form overproduce L-leucine and thus have a better nutritional value.

This application claims priority from U.S. Provisional Application No. 60/339,895 filed Nov. 30, 2001.

### BACKGROUND

Methods and compositions for making and using a feedback insensitive enzyme in the L-leucine biosynthetic pathway in plants are useful to overproduce L-leucine in plants. Mutated isopropylmalate synthase (IPMS) is an example of such an enzyme. Several forms of isopropylmalate synthase cDNA molecules were isolated, sequenced, characterized and their expression patterns analyzed. Mutations of these genes desensitize L-leucine negative feedback inhibition inherent in plants to enable overproduction of L-leucine in plants.

One of the important nutritional components of food plants is amino acid content. The higher the levels of the essential amino acids synthesized by a plant, the higher its nutritional value to humans and farm animals upon which humans feed directly or indirectly (e.g. eggs and milk). L-leucine is an important amino acid in food. In addition, L-leucine can be used as an additive in medical treatments, and pharmaceutical or chemical products.

Lysine, threonine, methionine, and isoleucine are members of the aspartate family of amino acids. Alanine, valine, and L-leucine are members of the pyruvate family of amino acids. Although they originate from two different starting metabolites, isoleucine, valine, and L-leucine are sub-classified into the branched-chain amino acid family because they are structurally and metabolically related (FIG. 1). Isoleucine and valine biosynthesis pathways share multiple enzymes and their metabolites are structurally similar. Threonine dehydratase/deaminase (TD), the first enzyme of a biosynthesis pathway leading to isoleucine converts threonine into 2-oxobutyrate. In the following steps the isoleucine and valine pathways share the activity of acetohydroxyacid synthase (AHAS), also known as acetolactate synthase, acetohydroxyacid reductoisomerase, dihydroxyacid dehydratase, and a transaminase. Biosynthesis of L-leucine involves the novel participation of isopropylmalate synthase (IPMS), isopropylmalate isomerase, and isopropylmalate dehydrogenase along with a transaminase. Regulation of the biosynthesis of the branched-chain amino acids is achieved by feedback inhibition of TD, AHAS, and IPMS by the sequential accumulation of end products. Accumulation of isoleucine inhibits the activity of TD allowing the shared enzymes to act on the metabolites of the valine pathway thus producing more valine and leucine (FIG. 2). As L-leucine levels increase, the end product inhibits the activity of IPMS and AHAS thus shunting the available metabolites toward valine production. Finally, increasing amounts of valine also inhibit AHAS to effectively shut down branched-chain amino acid synthesis.

In Arabidopsis thaliana, the sequences of the regulatory enzymes ALS/AHAS, and TD responsible for the biosynthesis of valine and isoleucine are known, and the activities of these enzyme have been reported. The branched-chain amino acid L-leucine is synthesized from 2-ketoisovalerate of the valine biosynthetic pathway, in a process that involves four distinct enzymes. The genes encoding these enzymes have been sequenced and characterized in bacteria and yeast, however they are not well characterized in plants. Therefore, identification, isolation and characterization of IPMS genes from plants are necessary to address some of the nutritive issues in food plants. Studies in bacteria, yeast, and some plant species have reported that IPMS is controlled by negative feedback inhibition in response to L-leucine levels. IPMS converts 2-ketoisovalerate (2-oxoisovalerate) into 2-isopropylmalate (3-carboxy-3-hydroxyisocaproate) at the junction where L-leucine biosynthesis branches from valine biosynthesis. Because IPMS has a regulatory role at a branch site of amino acid synthesis and it functions similarly across many taxa, it has maintained certain consensus regions that are needed for its activity and regulation of L-leucine biosynthesis.

In plants, no IPMS mutations are reported that lead to loss of L-leucine negative feedback inhibition and, consequently, overproduction of L-leucine. In yeast, there is a report on mutants selected for resistance to trifluoroleucine displaying an IPMS form that was insensitive to feedback control by L-leucine. IPMS genes from bacteria have been isolated and characterized, and mutations have been reported that affect the regulatory binding site leading to loss of L-leucine feedback sensitivity. A bacterial IPMS gene that is desensitized in the L-leucine feedback inhibition was reported in U.S. Pat. No. 6,403,342. The existence of codon bias prevents the bacterial genes from being expressed effectively when engineered in plants because of the low, or even sometimes lack of, the specific t-RNA types, in plants, that complement the bacterial codons for some amino acids. This results is very low expression or no expression of the bacterial gene in transgenic plants. In addition, IPMS enzymes of plants are encoded by nuclear genes and transcribed in the nucleus, but the mature enzymes are functioning in the chloroplasts. A bacterial IPMS protein does not have a chloroplast leader sequence that is required for transport into the chloroplast. Therefore, a plant IPMS mutant (desensitized to negative control by L-leucine) is needed for high levels of expression in transgenic plants. Thus, identifying and analyzing IPMS genes from plants are necessary steps to overproduce L-leucine and to increase a plant's nutritive value.

### SUMMARY

A method to increase levels of amino acids in food plants is to interfere with negative feedback inhibition in amino acid biosynthesis pathways. In L-leucine biosynthesis, the rate limiting step is the conversion of α-ketoisovalerate to α-isopropylmalate catalyzed by the enzyme IPMS. Therefore, transforming a host organism with a DNA encoding a mutant form of IPMS desensitized in the feedback inhibition system is an effective approach to overproduce L-leucine. The mutations are designed to reduce the binding capabilities for the negative inhibitor Leu, without affecting catalytic activity. For example, to overproduce free L-leucine in higher plants, transformation of the plant with an IPMS mutant gene encoding a feedback insensitive form of IPMS, is a possible step. However, the use of yeast mutant IPMS genes to transform food plants is not an optimal solution because plants have their own codon bias. Rather, mutant IPMS genes desensitized in the feedback inhibition in plants would be useful for this purpose.

cDNA molecules of the members of the gene family encoding IPMS were isolated using consensus data accumulated from amino acid alignments in a reverse genetics approach. A reverse genetics approach includes the steps of identifying and isolating a desired gene sequence based on the alignment of conserved regions among related protein sequences from different species.

The invention includes an isolated DNA molecule with a nucleotide sequence selected from the group consisting of GenBank accession numbers AF327647, AF327648, and AY049037.

An aspect of the present invention is the identification of a member of IPMS gene family, IMS2, whose expression pattern indicates that it is a housekeeping gene. “Housekeeping” genes are genes involved in cellular maintenance, e.g. by encoding proteins needed for basic cellular functions. An IPMS isoform IMS2, designated by GenBank accession number AF327648 is expressed to high levels in multiple tissues. Thus, IMS2 is a likely target for generating mutant forms of IPMS.

IPMS genes isolated and characterized as part of this invention can be used to generate mutant forms that are able to overcome the feedback inhibition by L-leucine and thus can accumulate L-leucine to higher than normal levels. Thus, nutritive value of plants can be enhanced but transforming them with mutant IPMS genes.

A method for enhancing the nutritional value of a plant includes the steps of:

a) obtaining a DNA molecule with a nucleotide sequence selected from the group consisting of GenBank accession numbers AF327647, AF327648, and AY049037;

b) mutating the DNA molecule wherein the mutated DNA encodes a protein having an isopropylmalate synthase activity with reduced feedback inhibition by L-leucine compared to a protein produced by a wild-type DNA molecule; and

c) transforming the plant with the mutated DNA, wherein the plant overproduces L-leucine compared to a non-transformed plant, therefrom has an enhanced nutrition value.

Other non-antibiotic selective markers are also suitable.

A method for overproducing L-leucine acid in a plant includes the steps of:

a) obtaining a DNA molecule with a nucleotide sequence selected from the group consisting of GenBank accession numbers AF327647, AF327648, and AY049037;

b) mutating the DNA molecule;

c) selecting the mutated DNA that encodes a protein having an isopropylmalate synthase activity with reduced feedback inhibition by L-leucine; and

d) transforming the plant with the mutated DNA to overproduce L-leucine.

A method for developing a plant genetic transformation marker includes the steps of:

a) obtaining a DNA molecule selected from the group consisting of GenBank accession numbers AF327647, AF327648, and AY049037;

b) mutating the DNA molecule;

c) transforming the mutated DNA into E. coli leucine auxotrophs;

d) selecting trifluoroleucine-resistant transformed E. coli cells; and

e) isolating the mutated DNA from the trifluoroleucine-resistant E. coli cells, said DNA molecule capable of being used as a plant genetic transformation marker.

Mutant IPMS alleles can be used as selectable markers in genetic transformation of plants by assaying for trifluoroleucine resistance. Other L-leucine analogs such as 4-aza-D-L-leucine or 3-hydroxy-D,L-leucine can also be used to select mutant IPMS alleles that are insensitive to feedback inhibition by L-leucine. Specific and random mutations are induced in isolated IPMS genes encoding the allosteric enzyme IPMS to reduce the binding capabilities for its negative inhibition by L-leucine, without affecting the enzyme's catalytic activity. These mutated enzymes lead to overproduction of L-leucine in plants when introduced into the plant's biosynthetic pathway, for example, by recombinant genetic methods known to those of skill in the art. These genetic markers are helpful in designing plant transformation vectors that are devoid of foreign bacterial antibiotic resistance genes such as genes that encode for kanamycin resistance. This is important considering that the presence of bacterial gene products in the food chain raises health safety issues.

Suitable transformation vectors are those capable of being transformed into bacteria, yeast, and plants. In plants, sequences to allow transport of IPMS with chloroplasts are provided.

An aspect of the invention includes a vector harboring a DNA molecule with a nucleotide sequence selected from the group consisting of GenBank accession numbers AF327647, AF327648, and AY049037, in particular a vector harboring a mutant form of DNA, wherein the mutant form is obtained by mutating a DNA molecule with a nucleotide sequence selected from the group consisting of GenBank accession numbers AF327647, AF327648, and AY049037. The mutant forms encode a protein having an isopropylmalate synthase activity with reduced feedback inhibition by L-leucine compared to a protein produced by a wild-type DNA molecule.

A cell transformed with a mutant form of the DNA, wherein the mutant form is obtained by mutating a DNA molecule with a nucleotide sequence selected from the group consisting of GenBank accession numbers AF327647, AF327648, and AY049037, encodes a protein having an isopropylmalate synthase activity with reduced feedback inhibition by L-leucine compared to a protein produced by a wild-type DNA molecule.

A seed transformed with a mutant form of the DNA is obtained by mutating a DNA molecule with a nucleotide sequence selected from the group consisting of GenBank accession numbers AF327647, AF327648, and AY049037. The mutant form encodes a protein having an isopropylmalate synthase activity with reduced feedback inhibition by L-leucine compared to a protein produced by a wild-type DNA molecule.

A plant transformed with a mutant form of DNA, wherein the mutant form is obtained by mutating a DNA molecule with a nucleotide sequence selected from the group consisting of GenBank accession numbers AF327647, AF327648, and AY049037, is a mutant form that encodes a protein having an isopropylmalate synthase activity with reduced feedback inhibition by L-leucine compared to a protein produced by a wild-type DNA molecule. Such a plant transformed with a mutant form of IPMS will over produce L-leucine, thereby increasing its nutritive value.

The spatial expression patterns of the different members of the IPMS gene family were analyzed in multiple tissues and organs of a higher plant, Arabidopsis thaliana. Arabidopsis is an acceptable model for food plants (or for any plant for that matter) acknowledged by those of skill in the art, because it has the same primary metabolism including the biosynthetic pathways of all amino acids. Enzymes catalyzing the steps of such biosynthetic pathways are highly conserved among higher plants in general.

The invention relates methods and compositions to increase levels of branched-chain amino acids in food plants. Food plants include all plants that are edible by humans and livestock. Examples of food plants include gymnosperms, rice, wheat, barley, rye, corn, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, clover, papaya, mango, banana, soybean, tobacco, tomato, sorghum, sugarcane, and alfalfa. Some of these plants are monocots, some dicots. Methods of transformation vary according to these types.

The phrase “mutant forms of IPMS genes” means that there are nucleotide changes from the wild-type genes that when encoded as proteins have lowered sensitivity to feedback inhibition by L-leucine, yet no significant reduction in their catalytic acitivity. The lowered sensitivity to feedback inhibition is determined by measuring the IPMS activity in the presence of varying amount of L-leucine and comparing the activity against the activity of wild-type or parent IPMS alleles.

The term “overproduce” for a specific transgenic plant that has been engineered with one of the IPMS-mutant forms mentioned in this patent means that the cell free levels of the amino acid L-leucine are synthesized at levels higher than those present in the non-engineered counterpart of the same plant species.

The term “transformed” describes both transient and stable genetic transformation of genes, by means of constructs, vectors, or naked DNA into host cells of plants and microorganisms such as bacteria, yeast by methods known to a skilled person in the art (see Materials and Methods).

Abbreviations

ABRC—Arabidopsis Biological Resource Center

AGI—Arabidopsis Genome Initiative

AHAS—acetohydroxyacid synthase

ALS—scetolactate synthase

BAC—bacterial artificial chromosome

BSA—bovine serum albumin

CGSC—Coli Genetic Stock Center

EST—expressed sequence tag

HPLC—High-Performance Liquid Chromatography

ILE—isoleucine

IPMS—isopropylmalate synthase

IPTG—isopropyl β-D thiogalactopyranoside

LEU—leucine

M-MLV-RT—Moloney Murine Leukemia Virus Reverse Transcriptase

MTR—L-O-methylthreonine

TAIR—The Arabidopsis Information Resource

TD—threonine dehydratase/deaminase

TFL—trifluoroleucine

RPA—RNA Protection Assay

VAL—valine

### BRIEF DESCRIPTION OF THE DRAWINGS

FIG. 1 shows the branched-chain amino acid biosynthetic pathways. The key regulatory enzymes that are regulated by end-product (negative) feedback inhibition are italicized and shown in bold. TD is inhibited by isoleucine, acetolactate synthase is inhibited by valine and leucine, and isopropylmalate synthase is inhibited by leucine.

FIG. 2 shows proposed sequential control mechanism for branched-chain amino acid biosynthesis as in Bryan (1990). Increasing levels of isoleucine inhibit threonine dehydratase/deaminase (TD)(2), which lowers levels of 2-oxobutyrate allowing acetohydroxyacid synthase (AHAS) to utilize pyruvate to produce valine and leucine. Increasing levels of leucine inhibits isopropylmalate synthase (IPMS)(3) as well as AHAS(3) shunting available metabolites toward valine production. As valine levels increase AHAS is given a double inhibitory signal (4).

FIG. 3 shows RPA III probe design. All probes were PCR-generated with restriction sites anchored in the primers. Arrows denote probe sequence 5′ to 3′ in direction of the arrow. Bold lines denote the pBluescript sequence generated during transcription. Transcription was carried out from the T7 promoter from a linearized plasmid. Expected sizes of RNA protected fragments are denoted below each construct. Probe 1 was cloned in XbaI and Kpn I, and then linearized with Sac I prior to transcription. Probe 5 was cloned in Sac I and Xho I, and then linearized with Sac I. Probe 3 (loading control eif4A, a transcription factor) was cloned with Xho I and Kpn I, and linearized with Xba I.

FIG. 4 shows regions of conserved domains in deduced amino acid sequences of four IPMS forms in Arabidopsis when compared to IPMS molecules from other organisms.

FIG. 5 shows multiple alignment of deduced amino acid sequences of Arabidopsis thaliana with known sequences from bacteria, yeast, and plant species. Each panel represents one of the conserved regions used for sequence searches in GenBank. Numbers on the left indicate the residue number for each amino acid sequence. (A) illustrates region of alignment showing the conserved sequence TTLRDGEQ and PROSITE PS00815 (Hoffman et al., 1999); (B) illustrates region of alignment corresponding to the second highly conserved sequence NGIGERAGN; (C) illustrates the third region of alignment corresponding to the conserved sequence SGIHQDG.

FIG. 6 shows phylogenetic representation of sequences predicted by the program MegAlign using the neighbor-joining method of Saitou and Nei (1987); length of each pair of branches represents the distance between sequence pairs; numbers on the bottom scale indicate the number of substitution events within each branch. Numbers located at each branch represent the ancestral nodes; (A) is a phylogenetic tree representation of all four Arabidopsis thaliana IPMS nucleotide sequences aligned with sequences of all the species in the original alignment shown in FIG. 4; (B) is the phylogenetic tree of the amino acid sequences of IPMS from Arabidopsis thaliana and other species from the original alignment in FIG. 4.

FIG. 7 shows segments of nucleotide alignment used to design IMS probes for use in the ribonuclease protection assay (RPA); bold uppercase letters indicate exact match between sequences; lowercase letters indicate sequence disparity; dashes indicate gaps in nucleotide alignment; sequences from A have no similarity to sequences from IMS2 or IMS3; sequences from B have no similarity with IMS1 or F15H18; probe sequences covered the entire length of each lower sequence (F15H18 and IMS3): (A) nucleotide sequence of IMS1 and F15H18 aligned to discern region of high similarity; probe 1 was PCR generated using F15H18 as the template; probe 1 was designed to hybridize to both IMS1 and F15H18; the disparity at the end of the target sequence produced a large mismatch easily recognized by RNase, while the other minor mismatches were skipped; ribonuclease digestion produced a single protected fragment of 213 bp corresponding to IMS1; the expected 259 bp full-length protected probe produced by hybridization with F15H18 transcripts was not detected; (B) nucleotide sequences of IMS2 and IMS3 are aligned to find a similar region of high similarity ending in disparity; probe 5 was PCR generated with IMS3 as template; ribonuclease digestion produced two distinct bands: 120 bp corresponding to IMS2 and 184 bp the full-length probe corresponding to IMS3.

FIG. 8 illustrates the ribonuclease protection assay of IPMS sequences in Arabidopsis thaliana; total RNA from month old plants was isolated and hybridized with probe 1 (IMS1), probe 5 (IMS2 and IMS3), and a loading control probe; IMS1, IMS2, and IMS3 are the three Arabidopsis IPMS genes isolated; four tissues were assayed for the expression of IMS: R-roots, L-leaves, S-stems, and F-Flowers; Eif-4A is the elongation initiation factor-4A used as a loading control.

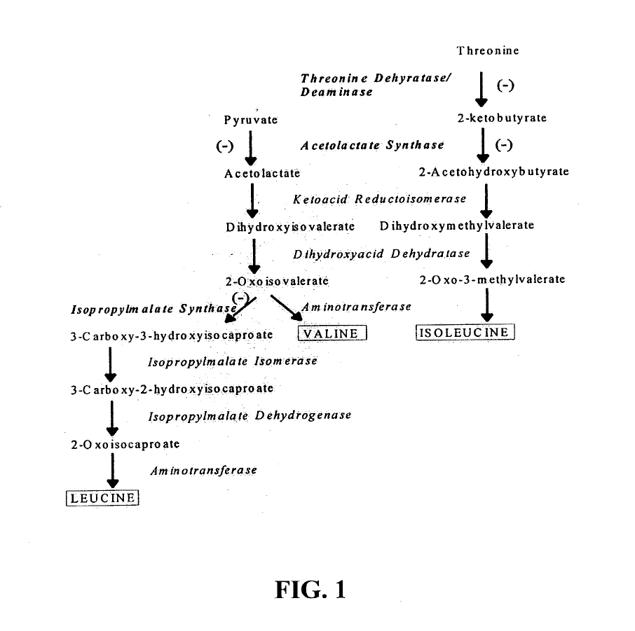

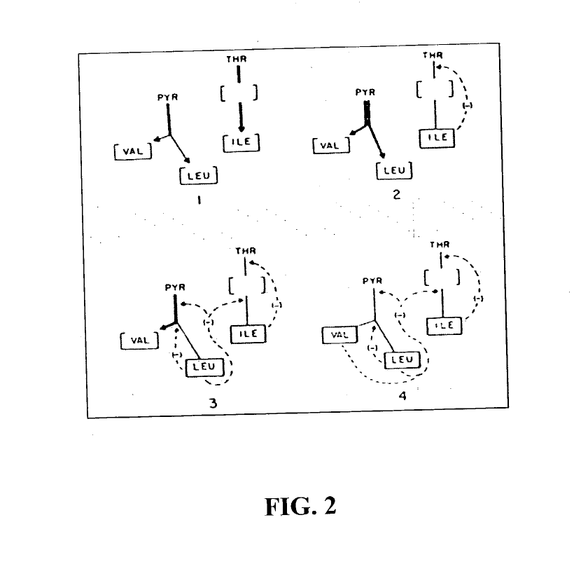

### DETAILED DESCRIPTION OF THE INVENTION

Full length cDNAs were isolated for three of four putative genes, two in chromosome 1 and two in chromosome 5, coding for isopropylmalate synthase in Arabidopsis thaliana. Using cDNA library screening and TR-PCR, full length cDNAs were isolated and sequenced for three of the four-member IPMS gene family of Arabidopsis. The full length cDNAs of three members are designated IMS1, IMS2, and IMS3 of the four member gene family of Arabidopsis IPMS (GenBank accessions #AF327647, AF3277648, and AY049037). The fourth locus of IPMS in Arabidopsis does not seem to be transcribed. Also, RNA protection assays (RPA) were used to determine the organ expression patterns in Arabidopsis for the three isolated IPMS genes. Table 1 summarizes the information about IPMS cDNA clones IMS1, IMS2 and IMS3, of the present invention and their accession numbers. Their corresponding BAC clones have been identified by random sequencing and deposited in GenBank by the Arabidopsis Gene Initiative (AGI) project after full length cDNA clones were isolated as part of the present invention. Expression studies have shown that IMS1 is expressed at very low amounts in roots, leaves, stems and flowers. IMS2 is highly expressed in leaves and roots and is expressed at lower levels in stems. IMS2 expression in flowers is very low. IMS3 is expressed in leaves and at a lower level in roots and stems. No expression of IMS3 was detected in flowers.

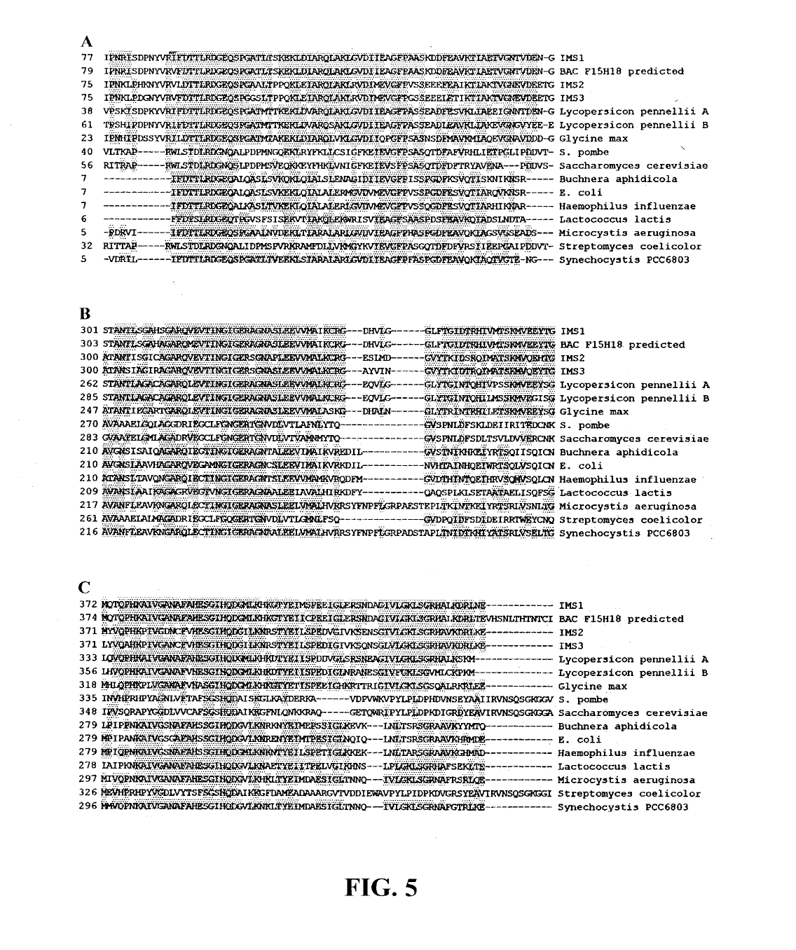

All three isolated clones and the predicted coding region of the fourth locus contain properties, both at the nucleotide and amino acid level, consistent with other IPMS sequences from other organisms (FIG. 5). All IPMS loci of Arabidopsis encode amino acid sequences that contain the three original conserved regions elucidated herein as well as the two documented PROSITE conserved regions PS00815 and PS00816 (Hoffman et al., 1999; Bucher and Bairoch, 1994). Sequence analysis has also shown the presence of chloroplast leader sequences at the N-terminal region of each of the four IPMS proteins. This would locate the mature IPMS protein in the chloroplast where it functions. The ability of IMS2 and IMS3 sequences to revert a leu(−) E. coli auxotrophic strain CV512 deficient in IPMS activity to prototrophy, reinforces the identity of the sequences isolated.

Functional complementation of E. coli leucine auxotroph strain VCV512 by Arabidopsis IMS genes was demonstrated. CV512 cells transformed with pTrc99A and plated on M9 medium supplemented with 60 μg/mL ampicillin and 20 μg/mL L-leucine grew. A plate with M9 minimal medium supplemented with 60 μg/mL ampicillin and 2 mM isopropyl β-D thiogalactopyranoside (IPTG) was prepared. CD512 cells grew upon transformation with recombinant pTrc99A containing truncated IMS3. CV512 cells grew upon transformation with recombinant pTrc99A containing truncated IMS2. As a control, CD512 cells transformed with vector pTrc99A with no inserts shows lack of growth confirming leucine auxotrophy and showing no reversion to prototrophy.

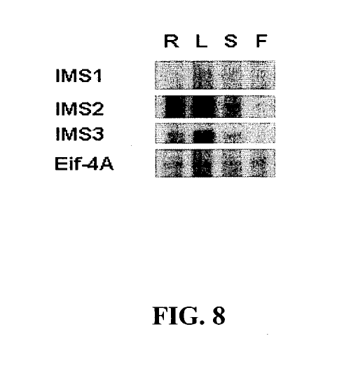

Expression analysis revealed that all three isolated clones are transcribed in a multitude of tissues (FIG. 8). The presence of the truncated EST clone 116C2T7 suggested that the fourth IPMS locus is indeed transcribed. However, library screening, RT-PCR, and RPA analysis in a variety of tissues did not isolate the cDNA of this fourth IPMS gene. Therefore, it may be a pseudogene, only transcribed under certain conditions not reproduced herein, or is transcribed at levels undetectable by current protocols.

TABLE 1:
IPMS cDNA CLONES

| Arabidopsis | IPMS1-1 | IPMS1-2 | IPMS5-1 | IPMS5-2 |
|---|---|---|---|---|
| IPMS form as | | | | |
| named by | | | | |
| Mourad Lab | | | | |
| Gene Locus | IMS1 | | IMS2 | IMS3 |
| name given by | | | | |
| Mourad Lab | | | | |
| Chromosome | 1 | 1 | 5 | 5 |
| Corresponding | BAC F2P9 | BAC F15H18 | BAC MYJ24 | BAC T2007 |
| BAC in | # AC016662 | # AC013354 | # AB006708 | # AB026660 |
| GenBank | | | | |
| Mourad Lab | IPMS1-200-C-1* | EST 116C2T7 | IPMS5-200-A-1* | IPMS5-600* |
| Clone # | | | | |
| Accession # of | # AF327647** | # T42657 | # AF327648** | AY049037** |
| cDNA clone | | | | |
| Nucleotides | 1896 | 2028 | 1512 | 1521 |
| long | | | | |
| Amino acids | 631 | 675 | 503 | 506 |
| long | | | | |
| MW (Daltons) | 68,135 | 73,525 | 55,212 | 55,124 |

*Isolated by the laboratory of Dr. Mourad.
**Deposited by the laboratory of Dr. Mourad.

To confirm the functionality of the Arabidopsis IPMS clones, the leucine auxotroph E. coli strain CV512 carrying a nonfunctional IPMS was transformed with a truncated version (missing most of the chloroplast leader sequence at the N-terminal end) of the Arabidopsis IMS genes. Truncated clones of IMS2 and IMS3 complemented the missing IPMS function in the auxotroph E. coli strain CV512.

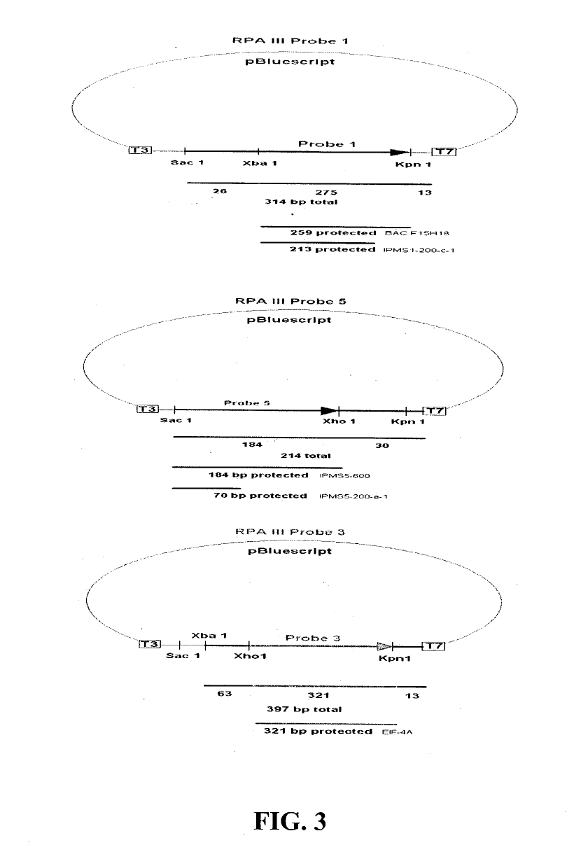

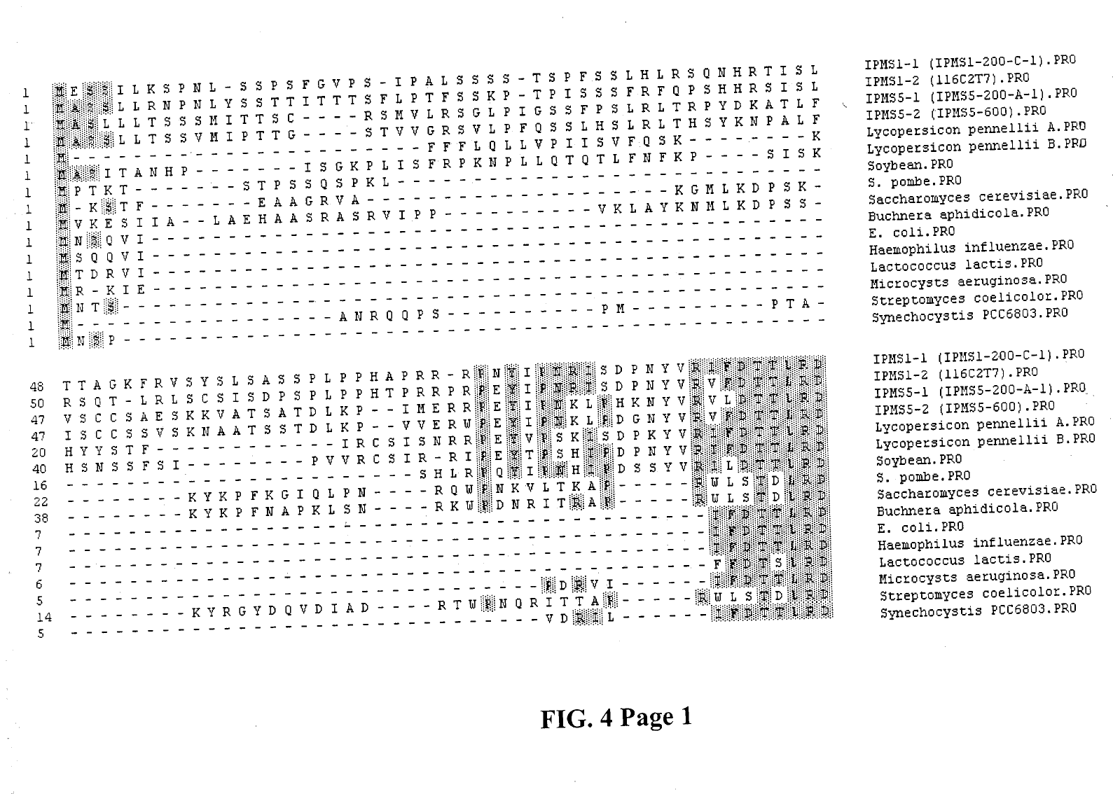

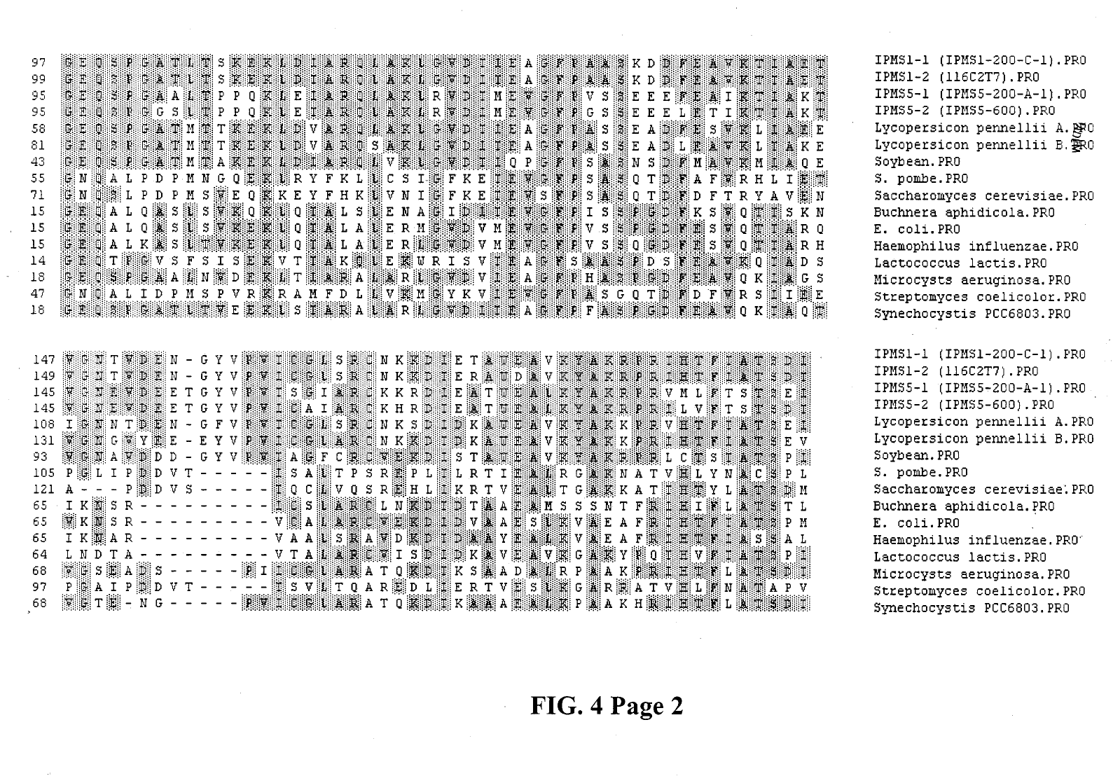

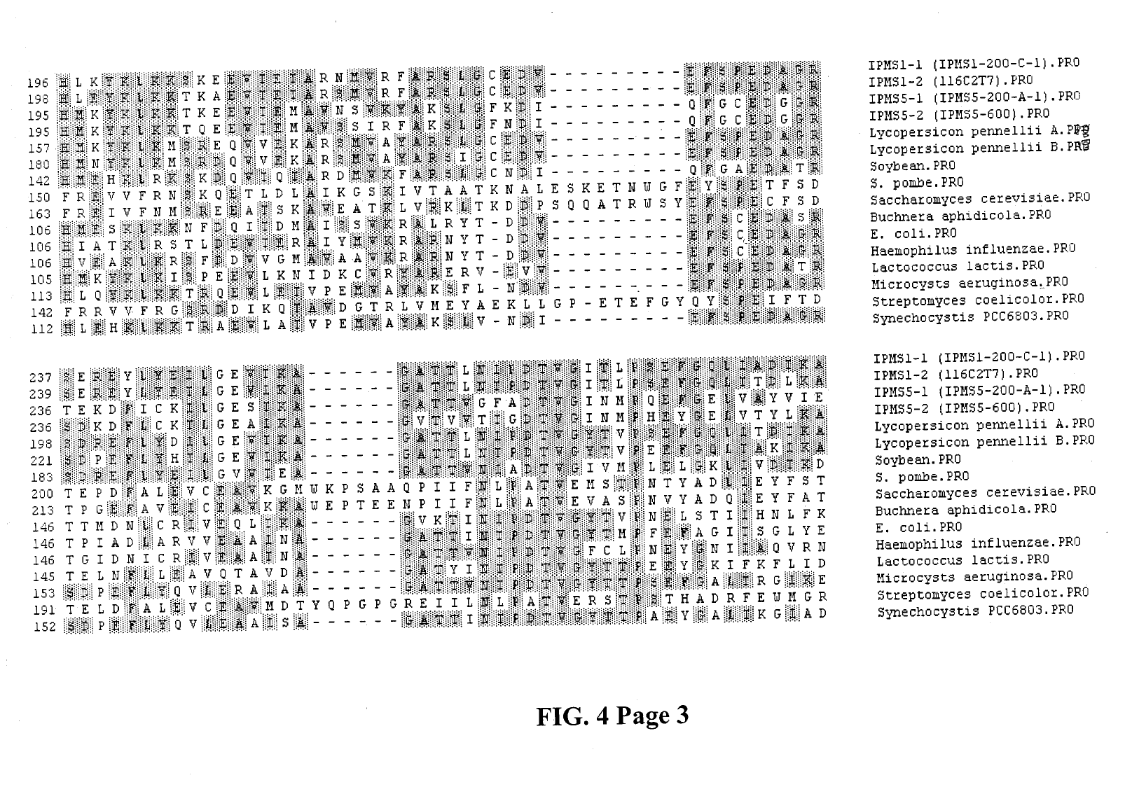

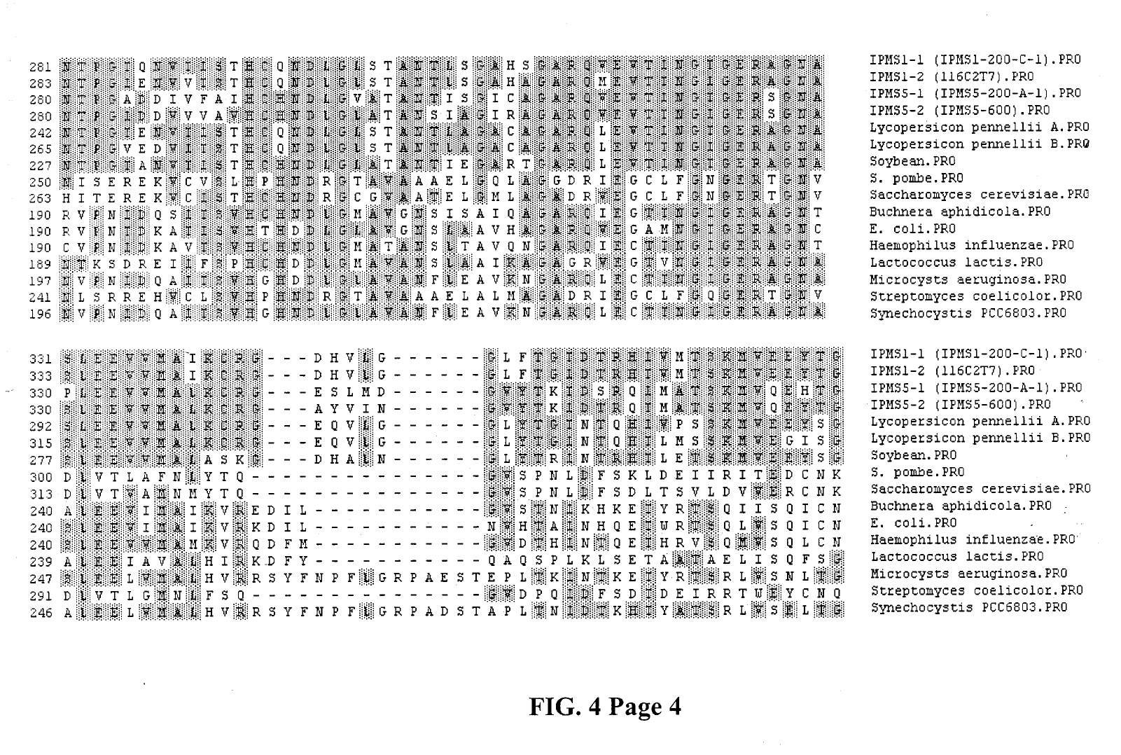

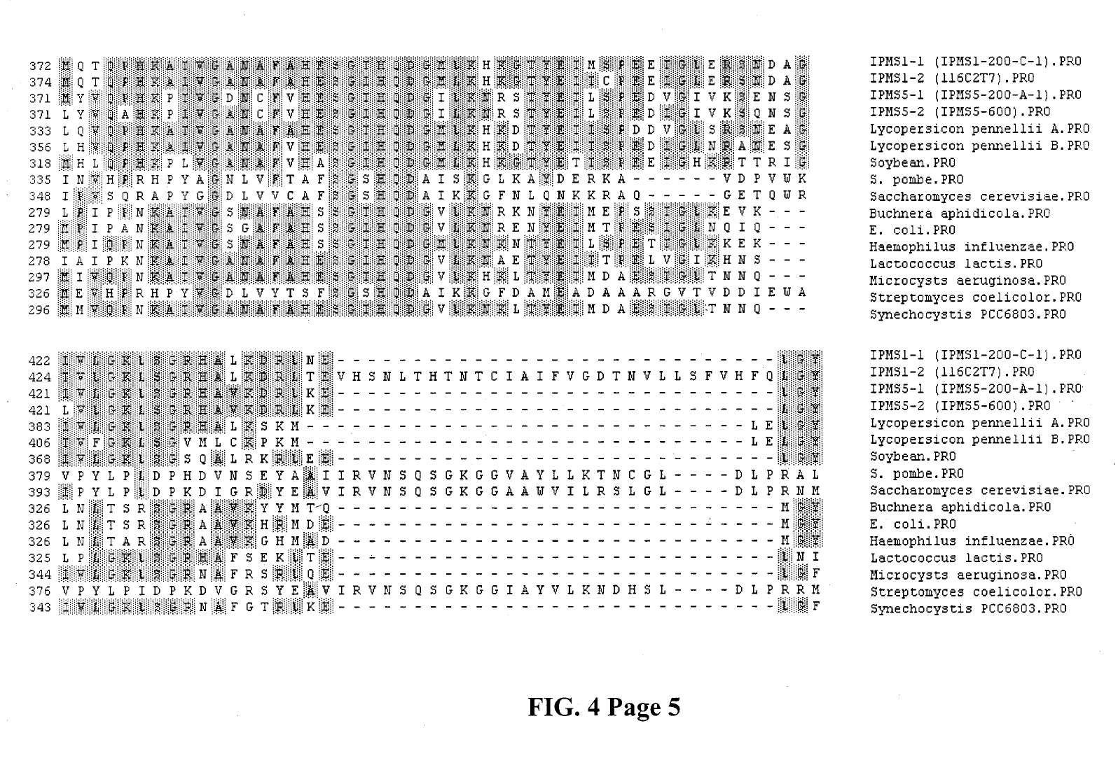

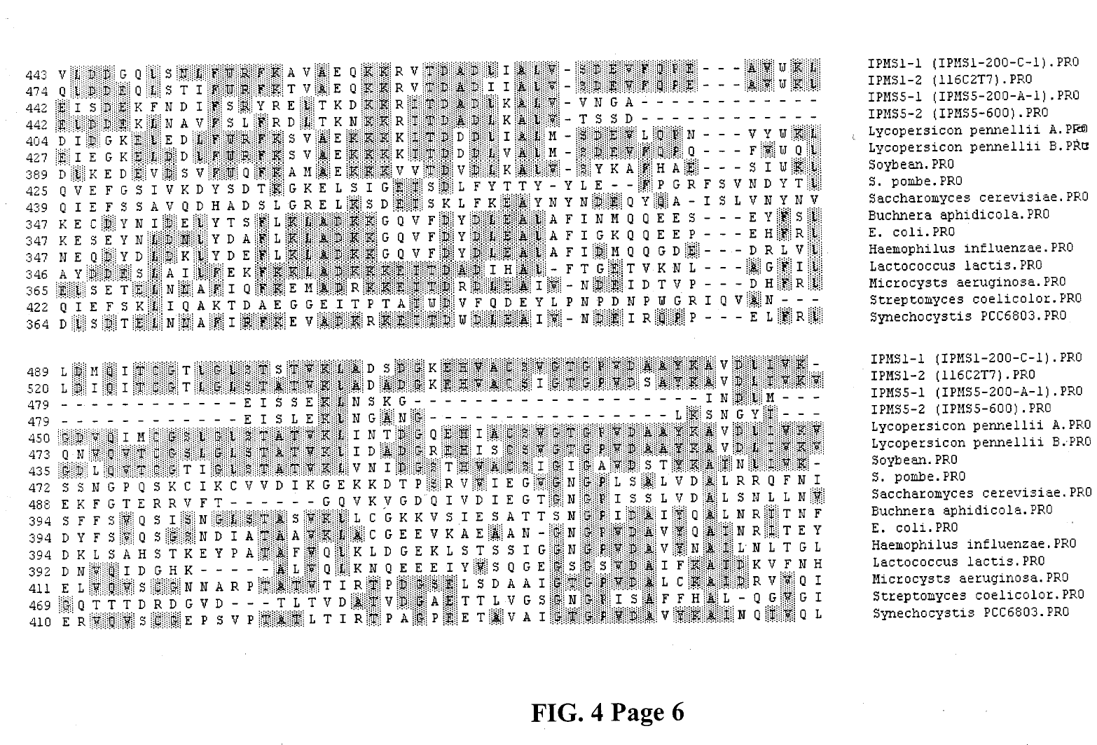

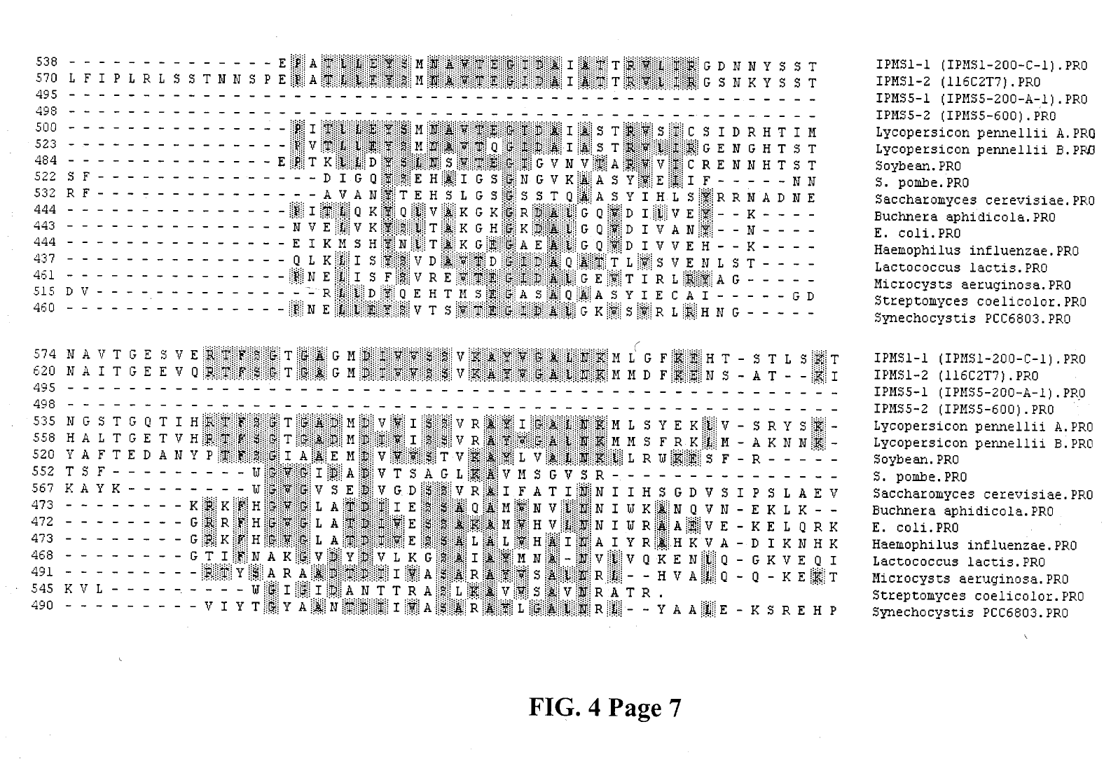

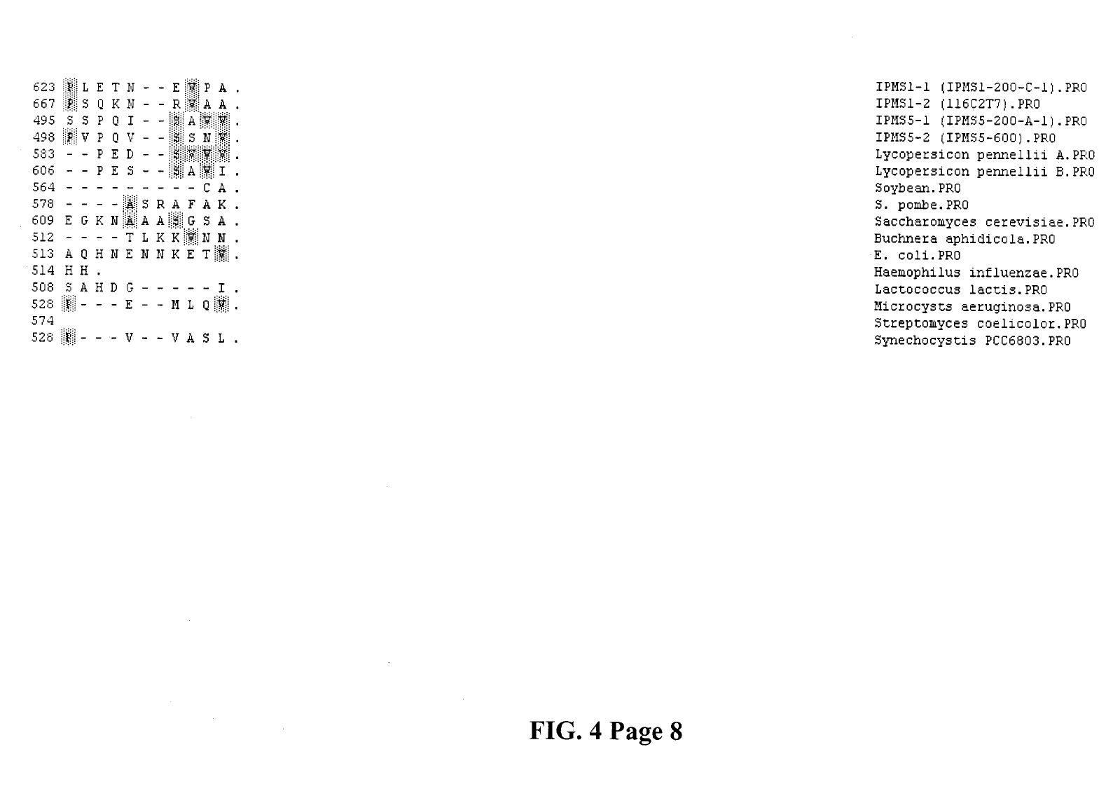

The expression of the four highly similar IPMS genes of Arabidopsis, were studied by using RNA protection assays (RPA). FIG. 3 describes the design of the RNA probes that were used in the expression assay. By analyzing protected RNA fragments from RNA (probe): RNA (cellular) hybridization experiments, it became apparent that IMS1 is expressed at very low amounts in all tissues tested while IMS2 is highly expressed in leaves and roots, but to a lesser extent in stems (FIG. 8). IMS3 was expressed in leaves and at a lower level in roots and stems. No expression of IMS3 was detected in flowers. IPMS1-2 does not seem to be expressed using the RNA protection assay experiments. This result is in agreement with failure to isolate this gene (IPMS1-2) by cDNA library screening or by RT-PCR.

Although multiple copies of IPMS are utilized in a few organisms, it is interesting that Arabidopsis has evolved a small family of isozymes to carry out only one function, because Arabidopsis has a simplified pathway organization and gene expression compared to other plants (Meyerowitz and Pruitt, 1985). Although production of leucine is critical and IPMS regulates the entire pathway, there may be some other underlying purpose for having so many highly homologous amino acid sequences. It is reported that branched-chain amino acid synthesis produces precursor molecules for elongation of branched short chain fatty acids (van der Hoeven and Steffens, 2000). Short and medium chain fatty acids constitute a wide variety of biomolecules as components of antibiotics, plant storage lipids, insect pheromones, and sugar polyesters secreted by Solanaceous plants as defenses against insect herbivores and pathogens (van der Hoeven and Steffens, 2000). In plants, the iso-branched short chain fatty acids 2-methylpropionic and 3-methylbutyric acids are derived from valine and leucine respectively by transamination and oxidative decarboxylation (Kandra and Wagner, 1990; Walters and Steffens, 1990; Luethy et al., 1997).

Another possible function of IPMS in plants concerns the biosynthesis of glucosinolates. Glucosinolates are thioglycosides that occur in Brassicaceae, in which the major class is derived from methionine (Campos de Quiros et al., 2000). Methionine-derived glucosinolates have serious biological and economic importance due to their degradation products, which include isothiocyanates, nitriles, epithiocyanates and thiocyanates (Bones and Rossiter, 1996). These degradation products have a multitude of bioactivities ranging from antinutritional to anticarcinogenic affects (Faulkner et al., 1997). Similar to sugar polyesters, glucosinolate degradation products also mediate plant herbivore interactions (Giamoustaris and Mithen, 1995). Reports of biochemical studies state that conversion of methionine to an alpha-ketoacid and subsequent elongation of the alpha-ketoacid by condensation with acetyl-CoA occurs prior to glucosinolate biosynthesis (Chisholm and Wetter, 1964). The condensation reaction of the alpha-ketoacid with acetyl-CoA is analogous to the first step of leucine biosynthesis catalyzed by IPMS (Campos de Quiros et al., 2000). The elongated alpha-ketoacid is either a substrate for 3C side chain glucosinolate formation, or is subject to another condensation reaction with acetyl-CoA to form 4C side chain glucosinolates (Campos de Quiros et al., 2000). Mendelian genes responsible for glucosinolate biosynthesis were located in chromosomes 4 and 5 in Arabidopsis thaliana (Magrath et al., 1994; Mithen et al., 1995). In addition, a quantitative trait locus (QTL) was mapped to a position in chromosome 5 coincident with the Mendelian GSL-ELONG locus (Campos de Quiros et al., 2000) that corresponds to the tandem repeat of IMS2 and IMS3 genes disclosed herein. Genes of the IPMS family recognize increasingly longer templates for acetyl-CoA condensation to produce elongated forms of methionine for the production of chain-elongated glucosinolates. This proposal would suggest, as does the model for fatty acid biosynthesis, that IPMS has the ability to recognize a variety of similar substrates that are utilized for chain elongation by the condensation of acetyl-CoA. The chain elongation of molecules by acetyl-CoA condensation plays a role in a variety of biosynthesis pathways. IPMS catalyzes this type of reaction to produce leucine in a negative feedback inhibition controlled reaction. It appears that in a similar fashion, members of the IPMS gene family catalyze the biosynthesis of other important molecules. Regulation of all of these pathways through IPMS would be a daunting task, since it is the first reaction in the production of these biomolecules. Because regulation of IPMS is a feedback inhibition by the end product L-leucine, control of free pools of amino acids would affect the amounts of multiple biomolecules. Therefore, it is intriguing to postulate that Arabidopsis harbors multiple forms of IPMS that have acquired diverging substrate specificity and regulatory reactions unique to each pathway. This would allow each isozyme to be regulated independently for each biosynthesis pathway.

It is also interesting to note the variable amount of sensitivity to L-leucine feedback inhibition displayed by the two members of IPMS in Saccharomyces cerevisiae. One isoform is highly sensitive to L-leucine levels while the other is not (Cavalieri et al., 1999). One possible explanation is that one IPMS member is directly responsible for the synthesis of L-leucine to be used in cellular protein synthesis, while the other member produces a basal level of IPMS activity to be utilized by the other pathways. In this example, one isoform of IPMS has been conserved to produce only L-leucine in a negative feedback inhibition loop for amino acid biosynthesis, while the other forms have slightly diverged in their substrate specificity allowing the unregulated production of precursor molecules for the other pathways. These pathways could then be controlled at a later step in the biosynthesis that does not impede the normal function of IPMS in L-leucine production. This model could explain the leakiness of yeast leucine auxotrophs that require the deletion of two loci for complete auxotrophy. Deletion of the major LEU4 locus would normally cause a leucine auxotroph, but divergent IPMS activity encoded by LEU9 that normally produces fatty acids or glucosinolates, retains the ability to recognize the original substrate 2-ketoisovalerate and form 2-isopropylmalate for subsequent conversion to L-leucine. In this model, the organism would not merely contain a redundant copy of IPMS but duplication products that have divergent substrate specificities would be able to recognize the original substrate. Thus, IPMS activity as it is understood for L-leucine production would be produced by a divergent relative to overcome the auxotrophy.

### EXAMPLES

### Example 1

IPMS Amino Acid Sequence Alignments.

Alignments of the amino acid sequences of IPMS from bacteria, yeast, and plants showed that there was great similarity among different taxa for IPMS (FIG. 5). After aligning, many areas of conservation became apparent including two well-documented PROSITE consensus patterns, PS00815: L-R-[DE]-G-x-Q-x(10)-K and PS00816: [LIVMFW]-x(2)-H-x-H-[DN]-D-x-G-x-[GAS]-x-[GASLI] (Hoffman et al., 1999;Bucher and Bairoch, 1994). The three regions corresponding to the amino acid sequences TTLRDGEQ (PS00815), NGIGERAGN, and SGIHQDG were used to search GenBank for related sequences in Arabidopsis thaliana.

The first search resulted in a partial EST clone OAO563 (GenBank accession #F13738 deposited Apr. 6, 1995) and a BAC clone T20O7 from chromosome 5 (GenBank accession #AB026660 deposited May 7, 1999). Because at the time it was not annotated, the BAC clone was analyzed by FgeneP software at the Baylor College of Medicine website www.searchlauncher.bcm.edu to elucidate any coding regions. The predicted coding region and the translated amino acid sequence were aligned with the known amino acid sequence of IPMS from other organisms. All three of the conserved regions were contained in the predicted coding region of the BAC T20O7. The partial EST clone in all frames was also translated and aligned with the other IPMS amino acid sequences. This showed that it contained the conserved region NGIGERAGN. Although both clones contained similarities at the amino acid level, they were quite different at the nucleotide level. These differences suggested that the EST OAO563 was not transcribed from the BAC T20O7 genomic sequence. Because the Arabidopsis Genome Initiative (AGI) was underway, searches of GenBank were conducted regularly with the short amino acid sequences and the nucleotide sequence of the EST OAO563. A search with the EST sequence matched a new BAC clone deposited Nov. 9, 1999 in GenBank. BAC clone F15H18 from chromosome 1 (GenBank accession #AC013354) was annotated and a coding region was predicted which upon translation also contained all three previously mentioned conserved regions. The coding region also matched the EST sequence at the nucleotide level.

### Example 2

Identification and Isolation of Full Length cDNAs of Three IPMS Genes.

The predicted coding sequences from BAC F15H18 from chromosome 1 and BAC T20O7 from chromosome 5 were used to prepare probes 1 and 5 respectively for cDNA library screening. Primary screening of Arabidopsis cDNA library CD4-15 with probe 1 identified one positive clone, IPMS1-200-C-1, which was sequenced, analyzed, and compared at the nucleotide level to the BAC clone F15H18. The sequences were extremely similar but they were not identical. A BLAST search of GenBank with the newly isolated cDNA sequence matched exactly an annotated BAC clone F2P9 deposited Oct. 5, 2000 on chromosome 1 (GenBank accession no. AC016662). Screening cDNA library CD4-14 with probe 5 identified one positive clone, IPMS5-200-A-1, which was also sequenced, analyzed, and compared at the nucleotide level to the BAC clone T20O7. They too were similar, but not identical. A BLAST search of GenBank with this cDNA sequence matched an annotated BAC clone MYJ24 deposited Dec. 27, 2000 on chromosome 5 (GenBank accession no. AB006708). At this time, the BAC clone T20O7 was annotated, and it was apparent there were four putative sequences that were similar to amino acid sequences of IPMS from other organisms. Attempts to isolate the remaining two sequences by screening cDNA libraries were unsuccessful. A CD4-7 a λ PRL2 cDNA expression library of Arabidopsis thaliana was also screened to ensure that the clones were not missed because of tissue or developmental specific expression. However, no further screening was successful. Nucleotide and amino acid sequences from all four BAC clones were used to search GenBank for full length ESTs for the remaining two clones. Although ESTs were found that matched the BACs, none were full length. Because the two sequences isolated from library screening matched the annotation of the BAC clones in GenBank exactly, the predicted coding regions from BACs F15H18 and T20O7 were used to design primers for use in RT-PCR to generate the remaining two cDNA clones.

RT-PCR produced the desired 1521 bp fragment from primers designed to amplify the entire coding region of BAC T20O7. Sequencing verified that the fragment was indeed the full length coding region represented by BAC T20O7, and the clone was named IPMS5-600. Attempts to RT-PCR generate the remaining clone using RNA isolated from a variety of tissues at different developmental stages have been unsuccessful. The two clones isolated by library screening and the single clone isolated by RT-PCR have been fully sequenced and deposited in GenBank: IPMS1-200-C-1 (IMS1) accession no. AF327647, IPMS5-200-A-1 (IMS2) accession no. AF327648, and IPMS5-600 (IMS3) accession no. AY049037.

### Example 3

Genome Organization and Similarity Among Arabidopsis IPMS Genes.

Analysis of the putative sequences with each other and other IPMS sequences of other organisms showed a large amount of sequence similarity (Table 2). At the nucleotide level, IMS1 and BAC F15H18 (both of chromosome 1) were 79.5% similar while IMS2 show 79.7% similarity to its chromosome 5 counterpart IMS3 (Table 2). However, nucleotide similarity between loci on different chromosomes is only about 50% (Table 2). Similarity at the protein level follows the chromosome-grouping trend as well. IMS1 is 83.4% similar to BAC F15H18, while IMS2 is 76.8% similar to IMS3 (Table 2). Again, protein similarity between loci on different chromosomes is only about 50% (Table 2). Phylogenetic tree construction showed that the chromosome 1 copies of IPMS are more similar to other plant sequences while IPMS copies of chromosome 5 seem to have diverged much earlier. This is evident at both nucleotide and protein sequence levels (FIG. 6). Table 3 outlines the major properties of the four loci. Much of the differences among the four Arabidopsis thaliana IPMS sequences are due to differences in their respective length. IMS2 is the shortest at 1512 bp. Translation of IMS2 predicts a 503 amino acid protein with a molecular weight of 55,212.54 Daltons, a 7.041 isoelectric point, and a 0.167 charge at pH 7.0 (Table 3). IMS3 is 1521 bp translated to a 506 amino acid sequence with a molecular weight of 55,124.5 Daltons, a 7.116 isoelectric point, and a 0.517 charge at pH 7.0 (Table 3). IMS1 is 1896 bp translated to a 631 amino acid sequence with a molecular weight of 68,135.34 Daltons, a 6.171 isoelectric point, and a −7.562 charge at pH 7.0 (Table 3). The predicted coding region from BAC F15H18 is the largest at 2028 bp translated to a 675 amino acid sequence with a molecular weight of 73,525.75 Daltons, a 6.521 isoelectric point, and a −4.322 charge at pH 7.0 (Table 3).

Translated amino acid sequences were analyzed for the presence of a signal peptide to elucidate sub-cellular localization of Arabidopsis IPMS using the network method published by Emanuelsson et al., 1999. All of the four Arabidopsis IPMS have a chloroplast leader sequence of slightly variable length at their N-terminal end with 46 amino acids long for IMS1, 51 amino acids long for IMS2, 49 amino acids long IMS3 and 57 amino acids long for the predicted coding region of BAC F15H18. The four loci were predicted to contain no other signal peptides.

Because the entire Arabidopsis thaliana genome has been sequenced and the BACs have been arranged by chromosome, it is possible to locate each clone within the genome (The Arabidopsis Information Resource (TAIR), www.arabidopsis.org/servlets/mapper, on www.arabidopsis.org). However, determining boundaries is novel and requires planning to identify desired functional regions. IMS1 is localized to the short arm of chromosome 1 while the partial EST116C2T7 corresponding to BAC F15H18 resides in the long arm of chromosome 1. IMS2 and IMS3 are closely linked to each other in the short arm of chromosome 5. Having multiple copies of IPMS gene sequences within the genome is not a characteristic unique to Arabidopsis. Both Saccharomyces cerevisiae (Chang et al., 1984), and Lycopersicon pennellii have multiple copies of IPMS (Wei et al., 1997). Studies in Saccharomyces cerevisiae have shown that in leu4 mutants, which are defective in one of two IPMS genes, IPMS activity was still detectable (Baichwal et al., 1983). Two loci must be interrupted in order to completely disrupt IPMS activity in yeast (Baichwal et al., 1983).

### Example 4

Functional Complementation of E. coli Leucine Auxotroph by IPMS Sequences.

To confirm the functionality of the Arabidopsis IPMS clones, the leucine auxotroph E. coli strain CV512 carrying a nonfunctional IPMS was transformed with a truncated version (missing most of the chloroplast leader sequence at the N-terminal end) of the Arabidopsis IMS genes. Truncated clones of IMS2 and IMS3 complemented the missing IPMS function in the auxotroph E. coli strain CV512. Transforming the E. coli leucine auxotroph strain CV512 lacking IPMS activity with a truncated version of a molecule lacking the chloroplast leader sequence of IMS1 produced two prototrophic colonies on M9 minimal medium. These colonies grew well upon subculturing on M9 minimal medium. However, these transformation results with IMS1 were not reproducible. It appears that the bacteria are able to synthesize the truncation transcript and ultimately a functional protein at very low levels. Both truncation constructs of IMS2 and IMS3 were able to complement strain CV512 and produced several prototrophic colonies. Upon further subculturing on M9 medium, these prototrophic transformants grew well. Secondary and tertiary attempts at transformation with the truncated versions of IMS2 and IMS3 consistently produced several prototrophic transformants. These results confirmed that the truncated Arabidopsis IPMS sequences are indeed expressing IPMS activity.

### Example 5

Spatial Expression Patterns of IPMS in a Higher Plant.

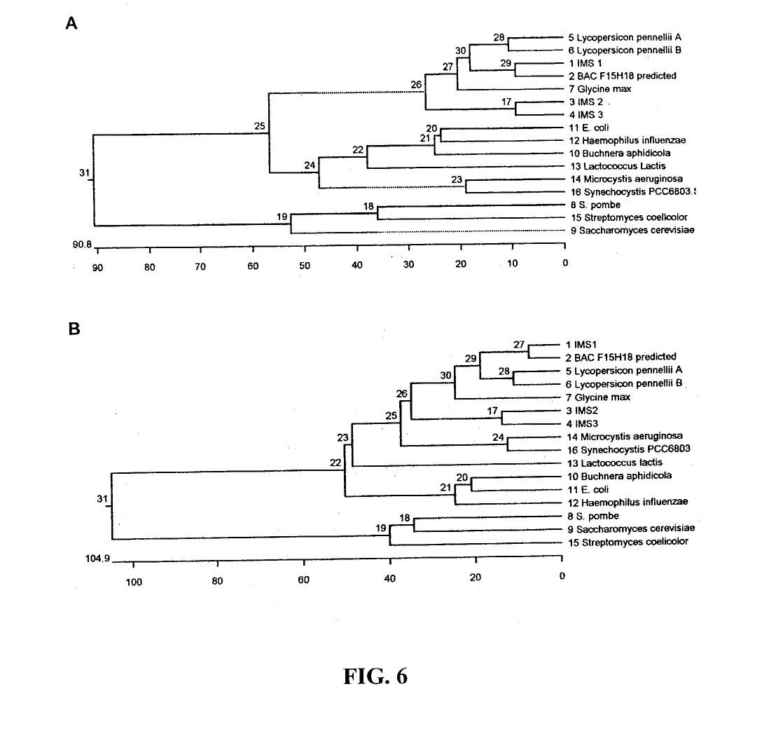

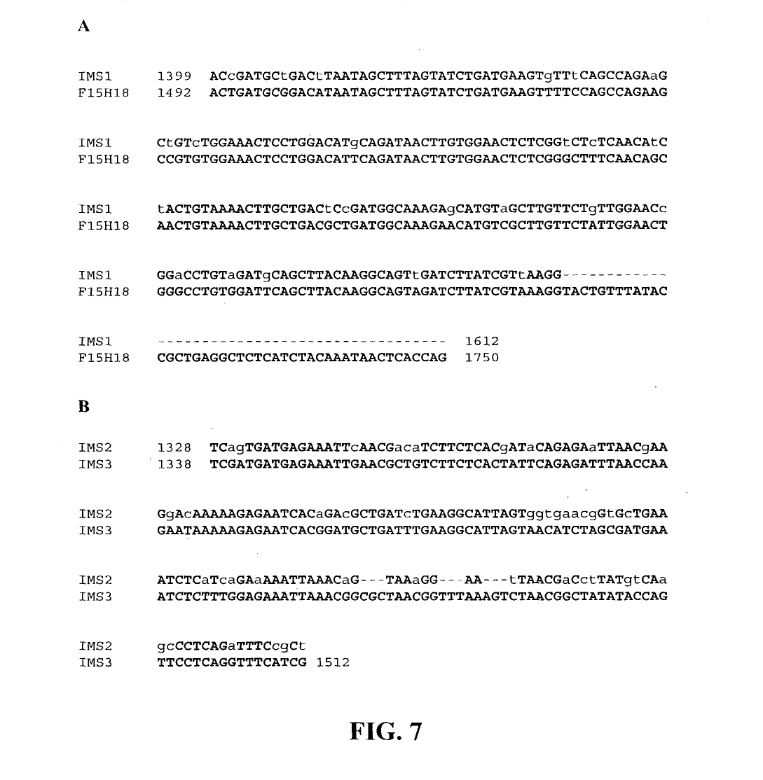

Because the four loci coding for Arabidopsis IPMS are highly homologous, it was difficult to devise a scheme to assay their individual expression. Results from the library screening already proved a tendency for probes to cross hybridize with all sequences of IPMS. It became clear that assaying the individual expression patterns of the Arabidopsis IPMS genes, using traditional Northern blot hybridization would not work. Unfortunately there were no areas long enough of non-similarity unique to each sequence that could be targeted for probing. Therefore, specific probing of each individual sequence would be impossible. However, sequence analysis showed that the four sequences could easily be categorized into two subsets by chromosome location (Table 3). Those sequences in chromosome 1 were highly homologous to each other, but not as homologous to the sequences on chromosome 5. Also, the sequences in chromosome 5 were highly homologous to each other, but not as homologous to the sequences on chromosome 1 (Table 2). Therefore, probe design was considered for the two groups separately. Unfortunately, cross hybridization between a probe and the two sequences of its specific subset was still a concern. Thus, two probes, one for each chromosome group, were designed that would bind both sequences from its subset. The two probes were designed to hybridize to each of the two sequences from its subset, such that it spanned an area of high sequence similarity and ended after an area of no sequence similarity between members of the pair (FIG. 7). RNase would cleave that area of no sequence similarity because it forms a hairpin in an RNA-RNA double strand molecule. Utilizing a ribonuclease protection assay, all four clones were discerned because the protected fragments varied in size by design. Probe 1 was designed from EST116C2T7 and spanned 259 bp. This probe also bound IMS1, but ribonuclease digestion produced a protected fragment of 213 bp (FIG. 7). Probe 5 was designed from IMS3 and spanned 184 bp. This probe also bound IMS2, but ribonuclease digestion produced a protected fragment of 120 bp (FIG. 7). Therefore, upon digestion all four sequences coding for IPMS were discernible: 259 bp for EST116C2T7, 213 bp for IMS1, 184 bp for IMS3 and 120 bp for IMS2.

IMS1 transcripts were found in very small amounts in roots, leaves, stems, and flowers (FIG. 8). IMS2 transcripts were detected in higher amounts in roots, leaves, and stems (FIG. 8). It was also expressed in very small amounts in flowers (FIG. 8). IMS3 transcripts were also expressed at higher levels in roots, leaves, and stems, but not expressed in flowers (FIG. 8). The predicted coding sequence from BAC F15H18 was not identified by the presence of the 259 bp protected fragment in any tissue assayed.

TABLE 2
Nucleotide and amino acid residue similarity of IPMS sequences
computed by the MegAlign program (Higgins and Sharp 1989)

| | Nucleotide and Amino Acid percent similarity | | | | | | | | | | | | | | |
|---|---|---|---|---|---|---|---|---|---|---|---|---|---|---|---|
| | F15H18^{a} | IMS2 | IMS3 | LpA | LpB | Gm | Sp | Sac | Ba | Ec | Hi | Ll | Ma | Stc | SP |
| IMS1 | 79.5 | 51.2 | 52.0 | 65.6 | 62.9 | 61.6 | 24.9 | 26.1 | 36.1 | 33.4 | 36.8 | 36.4 | 39.5 | 22.2 | 43.6 |
| | 83.4 | 52.6 | 53.8 | 72.5 | 68.7 | 66.1 | 17.8 | 18.9 | 37.8 | 41.4 | 42.2 | 40.7 | 50.0 | 18.1 | 52.4 |
| F15H18 | | 50.1 | 49.8 | 65.4 | 64.4 | 62.5 | 25.1 | 26.6 | 36.9 | 35.0 | 37.7 | 34.0 | 36.7 | 21.6 | 41.1 |
| | | 52.6 | 53.8 | 70.0 | 67.4 | 63.8 | 17.4 | 18.7 | 33.3 | 36.8 | 36.8 | 33.7 | 47.0 | 18.8 | 48.3 |
| 1M52 | | | 79.7 | 46.8 | 47.6 | 46.4 | 21.8 | 24.3 | 31.5 | 26.6 | 31.6 | 27.3 | 30.0 | 17.5 | 30.0 |
| | | | 76.8 | 48.8 | 48.4 | 46.2 | 17.7 | 16.9 | 31.0 | 31.2 | 33.9 | 31.0 | 36.9 | 16.1 | 38.1 |
| 1M53 | | | | 48.3 | 47.9 | 47.3 | 21.1 | 22.6 | 28.9 | 27.9 | 29.3 | 26.4 | 31.2 | 18.3 | 29.2 |
| | | | | 48.7 | 49.1 | 46.9 | 16.8 | 16.4 | 32.1 | 32.5 | 34.3 | 32.3 | 38.3 | 15.8 | 39.8 |

^{a}Values are based on predicted coding region from GenBank annotation.
Letters correspond to sources of sequence (accession numbers in Materials and Methods): LpA, Lycopersicon pennellii A; LpB, Lycopersicon pennellii B; Gm, Glycine max; Sp, Schizosaccharomyces pombe; Sac, Saccharomyces cerevisiae; Ba, Buchnera aphidicola; Ec, Escherichia coli; Hi, Haemophilus influenzae; LI, Lactococcus lactis; Ma, Mycrocystis aeruginosa; Stc, Streptomyces coelicolor; SP, Synechocystis PCC6803.

TABLE 3
Sequence Information of Arabidopsis thaliana IPMS Loci

| | IMSI | IMS2 | IMS3 | ESTI 16C2T7 |
|---|---|---|---|---|
| Isolated clone | IPMS1-200-C-1 | IPMS5-200-A-1 | IPMS5-600 | NA |
| Accession # | AF327647 | AF327648 | AY049037 | T42657 |
| BAC clone | F2P9 | MYJ24 | T20O7 | F14H18 |
| Chromosome | 1 | 5 | 5 | 1 |
| cDNA length (bp) | 1896 | 1512 | 1521 | 2028^{a} |
| Protein length (A.A.) | 631 | 503 | 506 | 675 |
| Molecular weight^{b} (Da) | 68,135.34 | 55,212.54 | 55,124.5 | 73,525.75 |
| Charge^{b} | −.562 | 0.167 | 0.517 | −4.322 |
| Isoelectric point^{b} | .171 | 7.041 | 7.116 | 6.521 |

^{a}EST116C2T7 represents 1600 bp of the full 2028 bp predicted coding region of BAC F15H18.
^{b}Values predicted by DNASTAR software based on nucleotide sequence and subsequent translation.

Materials and Methods

A. Generation of IPMS Mutant Forms

Random mutagenesis by PCR is performed on the IPMS gene family. A selection scheme is used (described herein) to select a mutant form(s) of IPMS that is desensitized to L-leucine feedback control.

The present invention provides a method for selecting mutant forms of IPMS that are desensitized in the feedback inhibition by L-leucine. This method includes the steps of:

1. Cloning of an isopropylmalate synthase gene IMS2 in a prokaryotic expression vector. Other forms of isopropylmalate synthase genes such as IMS1 and IMS3 are also suitable.

2. Mutating cloned IMS2 by PCR using the technique of Xu et al., 1999

A random mutagenesis strategy to generate mutant forms of a desired gene as described in Xu et al., (1999) involves a two-step Mn-dITP PCR method (dITP stands for 2′-deoxy-inosine 5′-triphosphate). The presence of Mn²⁺ and dITP induces base changes in the target sequence. The desired gene to be mutated for e.g., a truncated version (lacking chloroplast leader sequence) of an IPMS gene of the present invention is cloned in a suitable template plasmid. The plasmid is then subjected to two successive Mn-dITP PCR cycles. In the first PCR, 40 μM of Mn²⁺, 100 ng of template plasmid DNA, 5 pmol of each primer (gene specific or vector specific), 2 mM of Mg²⁺, 200 μM of dNTP and 5 U of Taq DNA polymerase in 50 μL are used. The PCR cycle starts at 94° C. for 3 min, followed by 20 cycles of 94° C. for 1 min and 72° C. for 1 min. The last cycle has an extension time of 10 min at 70° C. The extension time at 72° C. can vary depending on the length of the gene that needs to be mutated. Any standard thermocycler can be used. In the second PCR, 40 μM dITP, 2 μL mixture from the first PCR, same amount of primers, dNTP, Mg²⁺, and Taq DNA polymerase are used in 50 μL reaction for 30 cycles with same cycling conditions as the first PCR. The amplified PCR products are purified, digested with appropriate restriction enzymes and ligated into a suitable expression vector such as pET23a (+) (Novagen, Madison, Wis.). The ligated products are transformed into electrocompetent E. coli by electroporation. The transformants are analyzed by plating them on appropriate selection media. A desired transformant generated by the above method can be further analyzed by sequencing to identify the specific mutation(s) in the gene of interest. Depending upon the length of the gene used, a fully saturated random mutagenesis library can be obtained following the method described above.

3. Transforming the mutated plasmids into an IPMS auxotrophic E. coli strain CV512 as disclosed herein.

4. Selecting trifluoroleucine (TFL)-resistant transformed E. coli cells plated on a medium containing a toxic leucine analog, TFL. TFL exerts a toxic effect on cells because upon uptake from the medium it gets incorporated into cellular proteins in place of normal L-leucine, leading to cell death. Cells that are transformed with a mutant form of IPMS displaying desensitized negative feedback control will overproduce normal L-leucine and will be able to out compete the toxic TFL, bypassing its toxic effect. Other L-leucine analogs such as 4-aza-D-leucine, L-leucine or 3-hydroxy-D,L-leucine can also be used. Cells transformed with suitable IPMS mutations will grow and form colonies on plates supplemented with TFL while cells transformed with IPMS mutations that did not affect negative feedback control will not survive.

5. Subcloning the selected mutant forms into a plant transformation vector.

6. Transforming desired host plants with the plant transformation vector containing a mutant form of IPMS.

7. Selecting positive plant transformants by testing for resistance to trifluoroleucine (TFL) and confirming for L-leucine overproduction by analytical techniques such as High-Performance Liquid Chromatography (HPLC).

Selection of the desired mutant isoform is based on the level of desensitization to L-leucine feedback inhibition. Selection methodologies can include complementing an E. coli or yeast leucine autotroph with mutated IPMS gene sequences. Mutant allele(s) are transformed into wild type Arabidopsis and the transformants are analyzed for L-leucine overproduction. The selected IPMS mutant alleles are resistant to the toxic L-leucine analog trifluoroleucine (TFL) and thus are insensitive to feed back inhibition by L-leucine. Another value of the isolated mutant IPMS alleles is as selectable markers in genetic transformation of plants (TFL-resistance). Trifluoroleucine resistance conferred by LEU4 which encodes a leucine feedback insensitive IPMS was used as a dominant selectable marker in transformation of yeast strains isolated from wine (Bendoni et al., 1999). Arabidopsis mutant IPMS alleles provide a mutant plant IPMS marker that is useful as a dominant selectable marker for plant genetic transformation. Such plant genes will be expressed better than yeast genes when transformed into plants because codon bias in plants prevents optimal expression of non-plant codons.

B. Plant Material and Growth Conditions

Arabidopsis thaliana Columbia wild type seeds were planted in pots filled with moistened potting mix. Seeded pots were placed in 4° C. for two days before they were transferred to a growth chamber where they germinated and grew at 50% humidity and 20° C. with constant 24-hour fluorescent light until maturity and seed set.

C. GenBank Search for IPMS Sequences

A search of GenBank at the National Center for Biotechnology Information (www.ncbi.nlm.nih.gov) was conducted to identify as many known IPMS sequences as possible. Many sequences were found for bacteria and a few were found for yeast and plants. Amino acid sequences from Lycopersicon pennellii (Accession #O04973 and O04974), Glycine max (Accession #Q39891), Schizosaccharomyces pombe (Accession #CAA207723), Saccharomyces cerevisiae (Accession #P06208), Buchnera aphidicola (Accession #O31287), E. coli (Accession #P09151), Haemophilus influenzae (Accession #P43861), Lactococcus lactis (Accession #Q02141), Mycrocystis aeruginosa (Accession #P94907), Streptomyces coelicolor (Accession #AAB82586), and Synechocystis PCC6803 (Accession #P48576) were aligned using the MegAlign (DNASTAR Madison, Wis.) program utilizing the Clustal algorithm (Higgins and Sharp, 1989) with a PAM250 residue weight table (FIG. 5). Searches of all deposited Arabidopsis thaliana sequences using the conserved amino acid sequences elucidated by the amino acid alignment were conducted using tfastx3 software (Pearson and Lipman, 1988) at TAIR (The Arabidopsis Information Resource www.arabidopsis.org). This software searches all six reading frames of DNA sequences deposited in GenBank.

D. Genomic PCR Reactions for Probe Generation

Arabidopsis genomic DNA was extracted using the Dneasy Plant Kit of QIAGEN (Catalog No. 69104). PCR was conducted using Taq DNA polymerase (Promega), 0.2 mM dNTPs, 1.5 mM MgCl2, 1 μM each primer, 1 μg genomic DNA, and deionized water to 100 μL total volume. The mixture was first denatured at 94° C. for 3 minutes. Then the mixture was cycled 30 times each cycle consisting of 94° C. for 1 minute, 53° C. for 2 minutes, and 72° C. for 2 minutes. Following the 30th cycle, a final extension at 72° C. for 7 minutes was done to finish all extensions.

E. Library Screening and Isolation of Two Arabidopsis Full Length IPMS cDNA Clones IMS1 and IMS2

The first exon (largest predicted coding regions) of the coding sequence from the bacterial artificial chromosome (BAC) clones F15H18 and T20O7 (Accession #AC013354 and AB026660 respectively) were used to prepare primers for genomic PCR. Because there was similarity between the sequences of the two BAC clones, an alignment of the nucleotide sequence of the first exon was done as disclosed herein and primers were designed in regions of decreased similarity to ensure the production of specific probes for each of the two BAC clones. Amplification products were electrophoresed in 0.7% agarose in Tris-Acetate EDTA buffer, and the probe was isolated utilizing a QIAquick Gel Extraction Kit (Qiagen). Probe 1 was 622 bp in length and probe 5 was 381 bp. Based on the size of the cDNA from the predicted coding regions (2028 bp for BAC F15H18 and 1521 bp for BAC T20O7), probe 1 was used to screen the Arabidopsis cDNA expression library CD4-15 (2-3 kb inserts) (Keiber et al., 1993) and probe 5 was used to screen the Arabidopsis cDNA expression library CD4-14 (1-2 kb inserts) (Keiber et al., 1993). Both cDNA libraries were obtained from the Arabidopsis Biological Resource center (ABRC) at Ohio State University. CD4-14 and CD4-15 are both λ Zap II cDNA expression libraries consisting of cDNA generated from 3 day-old Arabidopsis thaliana seedling hypocotyls using oligo d(T) as primer. Each library was titered and plated according to the methods as in Sambrook et al., (1989). Plaque lifts were conducted using HyBond-XL membrane (Amersham). Plaque lifts were performed as follows: filters were placed very carefully on each plate with plaques for one minute. Filters were then peeled and placed with plaques facing upward in denaturing solution (0.5 N NaOH, 1.5 M NaCl) for 2 minutes, transferred to neutralizing solution (1.5 M NaCl, 0.5M Tris.Cl pH 7.4) for 5 minutes, then finally washed in 2×SSC for 30 seconds and allowed to air dry on clean Whatman filter paper. The above procedure was repeated exactly as above with a second filter, except it was applied to the plate with plaques for two minutes, twice the original time. After air drying, filters were baked at 80° C. for 1 hour. Radiolabelled probes were generated by random priming using the Prime a Gene Labeling System (Promega, Madison, Wis.), [α-32 P] dCTP (3000 Ci/mmol), and purified using nick columns (Pharmacia, N.J.). The plaque lifts were soaked in a prehybridization buffer of 6×SSC, 5×Denhardt's reagent, 1% SDS and 100 μg/mL Herring sperm DNA at 65° C. for 2 hours. For hybridization, the radiolabelled probe denatured by boiling for 5 minutes then immediately added to the hybridization solution, which replaced the prehybridization solution on the filters. Hybridization buffer consisted of the same contents as the prehybridization buffer, and the hybridization proceeded for 18 hours at 65° C. The next day lifts were washed twice for 5 minutes each on a rotary shaker with 7×SSPE, 0.5% SDS, and exposed to Kodak X-OMAT AR film (Eastman Kodak) at −70° C. overnight in a Kodak x-ray cassette. The next day the film was developed. Agar plugs corresponding to the positive plaques were placed in eluting buffer and the eluted phages were subjected to subsequent secondary and tertiary rounds of screening as described above. For each probe, a single positive plaque isolated from the tertiary round of screening was used in the ExAssist protocol of Stratagene(California) to grow in the SOLR strain of E. coli that harbored the pBluescript plasmid. This allowed the excision of the cDNA insert and its insertion into the pBluescript plasmid in one step. Upon isolation of putative clones from each library, the recombinant plasmid DNA was purified then sent out for automated sequencing at Indiana University medical school. The DNA sequences were analyzed, translated, and aligned with the sequences from the first alignment using the software package DNASTAR (Madison, Wis.).

F. RT-PCR to Generate IPMS Clone IMS3

RT-PCR primers were designed so that the 3′ end of the primer used in the reverse transcriptase reaction landed on nucleotides that were non-homologous among the four clones. This allowed the primer to bind to the specific cDNA of choice and its subsequent amplification. Total RNA was isolated from whole wild type plants at the rosette stage at the emergence of the sixth pair of leaves using the RNeasy Plant Mini Kit (Qiagen). Two micrograms of total RNA were incubated with 1 μg 3′ primer, 0.2 mM dNTPs, 25 units RNasin (Promega), and 200 units of Moloney Murine Leukemia Virus Reverse Transcriptase (M-MLV-RT) in a total volume of 25 μL for 60 minutes at 42° C. Two microliters of the newly synthesized first strand cDNAs were mixed with 50 pmol of each gene specific primer, 0.02 mM dNTPs, and Taq DNA polymerase (Promega). The reaction was denatured at 94° C. for 3 minutes, cycled 30 times with each cycle consisting of 94° C. for 30 seconds, 55° C. for 30 seconds, and 72° C. for 30 seconds, and finally extended at 72° C. for 7 minutes (Kawasaki et al., 1990). A band corresponding to 1521 bp was isolated from a 0.7% agarose gel utilizing a QIAquick Gel Extraction Kit (Qiagen). The fragment was cloned into the multiple cloning site of pBluescript using restriction sites anchored in the PCR primers. The insert was then sequenced commercially using the T7 and T3 promoters of pBluescript as sequencing primers to sequence the insert from both ends.

G. Subcloning Arabidopsis IPMS Genes in a Prokaryotic Expression Vector

Using the isopropyl β-D thiogalactopyranoside (IPTG)-inducible pTrc99A (Amann et al., 1988) expression vector (Pharmacia, N.J.), constructs were designed to express truncated versions of IMS1, IMS2, and IMS3 in the E. coli leucine auxotroph strain CV512 lacking IPMS activity (Somers et al., 1973), obtained from the Coli Genetic Stock Center (CGSC). Nco I sites (CCATGG) contain an ATG sequence that can be used for transcriptional starts. An Nco I site located in the multiple cloning site of pTrc99A downstream of RNA polymerase binding sites was used to clone fragments directionally in frame for transcription. Truncations of all isolated clones had been designed using PCR primers with an Nco I site anchored to the 5′ end of the primer that corresponds to coding sequence such that the very next codon contained the correct guanine residue needed to complete the Nco I recognition sequence. This allowed cloning of all three IMS sequences in frame into pTrc99A while altering the sequence only with the addition of a methionine at the beginning of the IPMS truncations. All truncations were designed to eliminate the predicted chloroplast leader sequence. After the inserts were cloned, transformation was carried out to introduce the expression vector to E. coli strain CV512. This strain grew well on M9 minimal medium (as in Sambrook et al., 1989) plates supplemented with L-leucine, and showed no growth on M9 minimal medium alone.

H. Preparation of Competent Bacterial Cells and Complementation of E. coli Leucine Auxotroph

E. coli CV512 cells were made competent by the CaCl2 method (Hanahan, 1983) and stored in aliquots at −70° C. Cells were thawed on ice and each of the three IMS truncations as well as the pTrc99A plasmid were added to separate aliquots and mixed gently. The mixtures were incubated on ice for 15 minutes then heat shocked at 42° C. for 45 seconds. After heat shock the cells were again incubated on ice for two minutes and then 500 μL SOC liquid medium (Sambrook et al., 1989) was added. Cells were incubated at 37° C. for 45 minutes with gentle shaking. After incubation all cells were spun down and washed 3 times with 1 mL M9 minimal medium. After washing of CV512, cells were placed in 400 μL final volume M9 liquid media and 200 μL aliquots were plated on two different plates. One plate contained M9, 60 μg/mL ampicillin, and 20 μg/mL L-leucine. The other plate contained M9, 2 mM IPTG, and 60 μg/mL ampicillin. Ampicillin was used to select for positive transformants. IPTG was used to induce high levels of expression of the IMS insert from the prokaryotic Trc promoter of the pTrc99A.

I. RNA Protection Assays (RPA) for Expression Studies

PCR was utilized to produce both probes 1 and 5 from partial EST 116C2T7 and IMS3 respectively. It was also used to produce probe 3 for loading control, in which genomic DNA was used as template since the probe was designed from an exon sequence of the elongation factor Eif-4A (Cheuk et al., 2000). Primers were designed with 5′ extensions that contained the proper restriction sites for subsequent directional cloning into plasmid pBluescript. PCR amplification products were electrophoresed in 2.0% agarose gels and the amplified DNA fragments were isolated from the gel using a QIAquick Gel Extraction Kit (Qiagen). Probe 1 was digested with Kpn I and Xba I, probe 3 was digested with Kpn I and Xho I and probe 5 was digested with Sac I and Xho I. Each probe was ligated into the corresponding sites in pBluescript using T4 DNA ligase (Promega) as per manufacturer's instructions. The ligation mixture was then used to transform competent E. coli DH5α cells. Positive colonies were then picked and the recombinant plasmid DNA was isolated using a QIAprep Spin Miniprep Kit (Qiagen). Plasmid DNA was linearized by Sac I for probes 1 and 5, and with Xba I for probe 3. These constructs allowed transcription of antisense RNA from the T7 promoter of pBluescript while incorporating some nonhomologous sequences from the multiple cloning site of the plasmid. Selecting a different restriction enzyme within the multiple cloning site for cloning and transcript generation, led to the production of a very short region of similarity (13 bp) among the different probes. This allowed the probes to be used simultaneously when hybridized to cellular RNA without template dependent probe-probe interaction.

J. RPA Probe Labeling

Radiolabelled antisense probes were produced using [α-32P] UTP (800 Ci/mmol) and a MAXIscript T7 Kit (Ambion, Texas). The probes were gel purified in 6% TBE-Urea precast gels (Invitrogen). Probes were excised from the gel with a scalpel and eluted using probe elution buffer from the RPA III Kit (Ambion). Total RNA was isolated from each tissue of Arabidopsis thaliana Columbia wild type using Plant RNA isolation aid (Ambion) and RNeasy Plant Mini Kit (Qiagen). RNA from flowers and stem tissues was isolated from one-month-old plants harboring mature siliques. Leaf and root tissues for RNA extraction were harvested from rosette plants at the emergence of the sixth pair of leaves. The nuclease protection assay was conducted using a RPA III Kit and protected RNA fragments were run on the same 6% TBE-Urea gels with RNA Century Marker (Ambion, Texas).

K. Cloning of Mutant Forms of IPMS in a Plant Transformation Vector and Plant Transformation

The plant transformation that is proposed here includes inserting one of the IPMS mutant genes to be developed into an Agrobacterium vector that is a derivative of the disarmed Ti plasmid. The IPMS mutant gene(s) will be cloned in front of the Cauliflower Mosaic Virus 35S promoter (which is constitutively expressed in higher plants) and the recombinant plasmid is transformed Agrobacterium tumefaciens. The latter is then used to transform the flower buds of Arabidopsis plants with the aid of vacuum infiltration. Once inside the plant cells, a region of the plasmid (called T-DNA for transfer DNA) is excised from the plasmid and inserted at random in one of the five Arabidopsis chromosomes (Bechtold N., et al.,1993).

Plant transformation strategies for monocots include particle bombardments of immature embryos, callus, cells, and cell lines with an expression cassette wherein a mutant form of IPMS gene is operably linked with a promoter (U.S. Pat. No. 6,281,411).

L. Transformations

Plan promoter regulatory elements from a wide variety of sources can be used efficiently in plant cells to express foreign genes. For example, promoter regulatory elements of bacterial origin, such as the octopine synthase promoter, the nopaline synthase promoter, the mannopine synthase promoter, and promoters of viral origin, such as the culiflower mosaic virus (35S and 19S), 35T (which is a re-engineered 35S promoter, WO 97/13402 published Apr. 17, 1997) and the like may be used. Plant promoter regulatory elements include ribulose 1-5-biphosphate (RUBP) carboxylase small subunit (ssu), beta-conglycmin promoter, beta-phaseolin promoter, ADH promoter, heat-shock promoters, and tissue-specific promoters.

Constitutive promoter regulatory elements may be used thereby directing continuous gene expression in all cell types at all times (e.g., actin, ubiquitin, CaMV 35S, and the like). Tissue specific promoter regulatory elements are responsible for gene expression in specific cell or tissue types, such as the leaves or seeds (e.g., zein, oleosin, napin, ACP, globulin, and the like) and these may alternatively be used.

Promoter regulatory elements may also be active during certain stages of the plants' development as well as active in plant tissues and organs. Examples of such include, pollen-specifc, embryo-specific, corn silk-specific, cotton fiber-specific, root specific, cotton fiber-specific, root-specific, seed endosperm-specific promoter regulatory elements. An inducible promoter regulatory element may be used for expression of genes in response to a specific signal, such as, for example, physcial stimulus (heat shock genes).

After the DNA construct of the present invention has been cloned into an expression vector, it is transformed into a host cell. A wide variety of plant tissues may be transformed during dedifferentiation using appropriate techniques described herein.

Transformation of a plant or microorganism may be achieved using one of a wide variety of techniques known in the art.

M. Dicot Transformation

A suitable method for transforming dicots plants include Agrobacterium-mediated floral dip and vacuum infiltration. An Agrobacterium tumefaciens strain carrying a binary vector is grown to saturation in Luria Bertani (LB) medium (Gibco-BRL, BRL, Carlsbad, Calif.) supplemented with an appropriate antibiotic. Cells are harvested by centrifugation and then resuspended in infiltration medium to a final optical density of approximately 0.80 prior to use. The infiltration medium includes 5.0% sucrose and 0.05% Silwet L-77 (OSi Specialties, Inc., Danbury, Conn. USA).

For a floral dip method, the plants are submerged in an Agrobacterium inoculum resuspended in the infiltration media. After a few seconds of gentle shaking, the plants are removed making sure the floral parts have contacted the inoculum. For vacuum infiltration, the plants are inverted into a beaker containing the Agrobacterium inoculum and a vacuum is applied using a vacuum pump for 2-3 min. The vacuum is released and the plants are removed from the beaker. Dipped or vacuum-infiltrated plants are placed in a tray covered with domes to maintain high humidity for 12-24 h after treatment. Domes are removed and the plants are allowed to set seeds. Seeds are collected and selected on appropriate media for transformants. The above mentioned methods are described in Clough and Bent (1998) and Bechtold and Pelletier (1998).

N. Monocot Transformation

A suitable method for delivering transforming DNA segments to plant cells is microprojectile bombardment. An example of biolistic method of transforming maize is described in U.S. Pat. No. 6,399,861. For biolistic transformation, carrier particles are coated with nucleic acids and delivered into plant cells by an accelarating force. Examples of carrier particles include those comprised of tungsten, gold, platinum, and the like.

Biolistic transformation is an effective means of stably transforming monocots. Susceptibility to Agrobacterium infection is not required. A method for delivering DNA into plant cells by acceleration is by a gene gun, which can be used to propel particles coated with DNA through a metallic screen, such as a stainless steel, onto a filter surface covered with plant cells cultured in suspension. A suitable gene gun includes model PDS-1000/He from Bio-Rad (Bio-Rad, Hercules, Calif.). The metallic screen disperses the particles to prevent large aggregates from damaging the recipient cells and also increases the frequency of transformation. For the bombardment, plant cells in suspension are preferably concentrated on filters or solid culture medium such as an agar dish. Alternatively, immature zygotic embryos or other target cells may be prepared on solid culture medium. The recipient plant cells to be bombarded are positioned at an appropriate distance below a macroprojectile stopping plate. In biolistic transformation, one may optimize the culturing conditions and the bombardment parameters to yield the maximum numbers of stable transformants of plant cells. Physical factors involve manipulating the DNA/microprojectile precipitate or those that affect the dispersal and velocity of either the macro- or microprojectiles. Biological factors include steps involved in manipulation of cells before and immediately after bombardment, the osmotic adjustment of target cells to help recover from the stress associated with bombardment, and also the nature of the transforming DNA, such as linearized DNA or intact supercoiled plasmids. It is believed that these manipulations are especially important for successful transformation of immature embryos as well. Usually several particle bombardments are carried out with different agar dishes containing isolated immature embryos. The embryos are removed from the agar plates and suitable conditions are provided for organogenesis. The plantlets are selected in appropriate selection media for identifying positive transformants. Gene expression and sequence analysis are performed to confirm stable genetic transformation events. Analytical methods such as PCR, and Southern blotting are helpful to analyze positive transformants.

O. Oligonucleotide Primers Used in the Isolation, Sequencing, and RNA Protection Assay of IMS1, IMS2, and IMS3 Full Length cDNAs

Isolation of IMS1 by cDNA Library Screening

IMS1 is the name given to the gene locus in chromosome 1 (one). The clone name in the inventor's lab is IPMS1-200-C-1. The full length cDNA sequence and its predicted encoded protein were deposited in GenBank by Dr. Mourad under the Accession #AF327647.

IMS1 was isolated as a full length cDNA clone by screening the Arabidopsis thaliana cDNA library CD4-15 obtained from the Arabidopsis Biological Resource Center (ABRC) at Ohio State University. The probe used for screening the library was a 622 bp PCR-amplified fragment from genomic DNA isolated of Arabidopsis thaliana, Columbia wild type, using the following pair of primers:

Right primer (5′ end primer): 5′-CCA CAC CTA TCT CCT CCT CTT-3′

Left primer (3′ end primer): 5′-CCT GCA TCT TCT GGA CTG AAC-3′

Isolation of IMS2 by cDNA Library Screening

IMS2 is the name of the second gene locus residing in chromosome 5 and coding for IPMS. The clone name in the inventor's lab is IPMS5-200-A-1. The full length cDNA sequence and its predicted encoded protein were deposited in GenBank by Dr. Mourad under the Accession #AF327648.

IMS2 was isolated as a full length cDNA clone by screening the Arabidopsis thaliana cDNA library CD4-14 obtained from the Arabidopsis Biological Resource Center (ABRC) at Ohio State University. The probe used for screening the library was a 381 bp PCR-amplified fragment from genomic DNA isolated of Arabidopsis thaliana Columbia wild type plants using the following pair of primers:

Right primer (5′ end primer): 5′-GTG GTT GGC CGG TCA GTG TTA-3′

Left primer (3′ end primer): 5′-CAC AGT CTT GGC GAT GGT CTT-3′

Isolation of IMS3 by RT-PCR (reverse transcription PCR)

IMS3 is the name of the third gene locus residing in chromosome 5 and coding for IPMS. The clone name in the inventor's lab is IPMS5-600. The full length cDNA sequence and is predicted encoded protein were deposited in GenBank by Dr. Mourad under the Accession number AY049037. The full length cDNA was isolated by RT-PCR using total RNA isolated from Arabidopsis thaliana Columbia wild type plants using the following pair of primers:

5′ end primer: 5′-CAG GTA CCA TGG CTT CAT CGC TTC TGA C-3′

3′ end primer: 5′-GGG AGC TCT TAC ACA TTC GAT GAA ACC TG-3′

Sequencing Primers

All three IMS genes, IMS1, IMS2 and IMS3, were sequenced first by using the T3 and T7 primers that prime the T3 and T7 promoters of the pBluescript vector and flanking the cDNA clone in each case. From the sequences produced by this first round of sequencing, internal sequencing primers were designed to finish sequencing the cDNA clone.

Internal sequencing primer for IMS1 was:

5′-TTA TCT GCA TGT CCA GGA GT-3′

Internal sequencing primer for IMS2 were:

5′-CAT CAG AGA TTC TCC TCG AC-3′

5′-CGA ATT CTT CCT CAG ACG AC-3′

Internal sequencing primer for IMS3 was:

5′-GAG GCC AAG GAT ACT CGT ATT CAC-3′

Truncation Primers Used for PCR Amplication of a Truncated Version of the IMS Genes to be Expressed in E. coli

5′ primer used for the truncation of IMS2 and IMS3

5′-CCA TGG TAT TAG ACA CGA CGC TTC-3′

3′ primer used for IMS2 PCR

5′-TCT AGA CGG CCG CTT TAT TCA TTA CA-3′

3′ primer used for IMS3 PCR

5′-GGG AGC TCT TAC ACA TTC GAT GAA ACC TG-3′ (same as the 3′ RT-PCR)

5′ primer used for the truncation of IMS1

5′-CCA TGG AGT CTT CGA TTC TCA AAA GC-3′

3′ primer used for IMS1 PCR

5′-TCT AGA GAT TTT CTT CAG GCA GGG AC-3′

Primers Used for Producing Probes to be Used in the RNA Protection Assay (Nuclease Protection Assay)

Probe 1 was designed to distinguish between the transcripts of IPMS genes located in chromosome 1, IMS1 and EST116C2T7 (the fourth gene member of the Arabidopsis IPMS gene family). Upon RNase treatment probe 1, produced a 259 bp protected fragment with transcript of EST116C2T7 and 213 bp protected fragment with the transcript of IMS1. The PCR primer pair that produced probe 1 were:

5′ primer (right primer): 5′-GCT CTA GAA CTG ATG CGG ACA TAA TAG C-3′

3′ primer (left primer): 5′-GCG GTA CCC TGG TGA GTT ATT TGT AGA T-3′

Probe 5 was designed distinguish between the transcripts of IPMS genes located in chromosome 5, IMS2 and IMS3. Upon RNase treatment probe 5, produced a 120 bp protected fragment with the transcript of IMS2 and 184 bp protected fragment with the transcript of IMS3. The PCR primer pair that produced probe 5 were:

5′ primer (right primer): 5′-GCG AGC TCC GAT GAT GAG AAA TTG AAC G-3′

3′ primer (left primer): 5′-GAC TCG AGC GAT GAA ACC TGA GGA ACT G-3′

Primers to amplify the probe used for protecting the transcript of the eukaryotic initiation factor 4A (eIF-4A) that was used as a loading control on the gel:

5′ primer (right primer): 5′-GCC TCG AGC TGA TGA GAA CGA AGA TG-3′

3′ primer (left primer): 5′-CCG GTA CCT ATC TGA GTC GCT TCT GC-3′

### DOCUMENTS CITED

Relevant sections of the Documents are incorporated by reference.

Amann, E., Ochs, B., and Abel K. J. (1988) Tightly regulated tac promoter vectors useful for the expression of unfused and fused proteins in Escherichia coli. Gene 69:301-315.

Baichwal, V. R., Cunningham, T. S., Gatzek, P. R., and Kohlhaw, G. B. (1983) Leucine biosynthesis in yeast. Curr. Genet. 7: 369-377.

Bechtold N., Ellis J., Pelletier G. (1993) In planta Agrobacterium mediated gene transfer by infiltration of adult Arabidopsis thaliana plants. C R Acad Sci Paris, Life Sciences 613: 1194-1199.

Bechtold, N., and G. Pelletier, 1998 In planta Agrobacterium mediated transformation of adult Arabidopsis thaliana plants by infiltration, pp. 256-266 in Arabidopsis Protocols, edited by J. M. MARTINEZ-ZAPATER and J. SALINAS. Humana Press, Clifton, N.J./Totowa, N.Y.

Bendoni, B., Caalieri, D., Casalone, E., Polsinelli, M., Barberio, C. (1999) Trifluoroleucine resistance as a dominant molecular marker in transformation of strains of Saccharomyces cerevisiae isolated from wine. FEMS Microbiol. Lett. 180:229-233.

Bones, A. M., and Rossiter, J. T. (1996) The myrosinase-glucosinolate system, its organisation and biochemistry. Physiol. Plant. 97: 194-208.

Bryan, J. K. (1990) Advances in the biochemistry of amino acids. In The Biochemistry of Plants, Vol. 16. Edited by Mifflin, B. J. pp. 161-195. Academic Press Inc., New York.

Bucher, P., and Bairoch, A. (1994) A generalized profile syntax for biomolecular sequences motifs and its function in automatic sequence interpretation. In ISMB-94; Proceedings 2nd International Conference on Intelligent Systems for Molecular Biology. Edited by Altman R., Brutlag D., Karp P., Lathrop R., Searls D. pp. 53-61. AAAIPress, Menlo Park.

Campos de Quiros, H., Magrath, R., McCallum, D., Kroymann, J., Scnabelrauch, D., Mitchell-Olds, T., and Mithen, R. (2000) α-Keto acid elongation and glucosinolate biosynthesis in Arabidopsis thaliana. Theor. Appl. Genet. 101: 429-437.

Casalone, E., Barberio, C., Cavalieri, D., and Polsinelli, M. (2000) Identification by functional analysis of the gene encoding α-isopropylmalate synthase II (LEU9) in Saccharomyces cerevisiae. Yeast 16: 539-545.

Cavalieri, D., Casalone, E., Bendoni, B., Fia, G., Polsinelli, M., and Barberio, C. (1999) Trifluoroleucine resistance and regulation of α-isopropylmalate synthase I in Saccharomyces cerevisiae. Mol. Gen. Genet. 261: 152-160.

Chang, L. L., Cunningham, T. S., Gatzek, P. R., Chen, W. J., and Kohlhaw, G. B. (1984) Cloning and characterization of yeast LEU4, one of two genes responsible alpha-isopropylmalate synthesis. Genetics 108:91-106.

Cheuk, R., Shinn, P., Brooks, S., Buehler, E., Chao, Q., Johnson-Hopson, C., Khan, S., Kim, C., Altafi, H., Bei, B., Chin, C., Chiou, J., Choi, E., Conn, L., Conway, A., Gonzalez, A., Hansen, N., Howing, B., Koo, T., Lam, B., Lee, J., Lenz, C., Li, J., Liu, A., Liu, J., Liu, S., Mukharsky, N., Nguyen, M., Palm, C., Pham, P., Sakano, H., Schwartz, J., Southwick, A., Thaveri, A., Toriumui, M., Vaysberg, M., Yu, G., Cavis, R., Federspeil, N., Theologis, A. and Ecker, J. (2000) Direct submission. Arabidopsis thaliana Genome Center, Department of Biology, University of Pennsylvania, 38^{th} and Hamilton Walk, Philadelphia, Pa. 19104-6018, USA.

Chisholm, M. D., and Wetter, L. R. (1964) Biosynthesis of mustard oil glucosides. IV. The administration of methionine-14C and related compounds to horseradish. Can. J. Biochem. 42: 1033-1040.

Clough, S. J. and A. Bent, 1998 Floral dip: a simplified method for Agrobacterium-mediated transformation of Arabidopsis thaliana.. Plant J. 16:735-743

Emanuelsson, O., Nielsen, H., and von Heijne, G. (1999) ChloroP, a neural network-based method for predicting chloroplast transit peptides and their cleavage sites. Protein Science 8: 978-984.

Faulkner, K., Mithen, R. F., and Williamson G. (1997) Selective increase of the potential anticarcinogen 4-methylsulphinylbutyl glucosinolate in broccoli. Carcinogenesis 19: 605-609.

Hanahan, D. (1983) Studies on transformation of Escherichia coli with plasmids. J. Molec. Biol. 166: 557-580.

Higgins, D. G., and Sharp, P. M. (1989) Fast and sensitive multiple sequence alignments on a microcomputer. CABIOS 5: 151-153.

Hoffman, K., Bucher, P., Falquet, L., and Bairoch, A. (1999) The PROSITE database, its status in 1999. Nucleic Acids Res. 27:215-219.

Kandra, L., and Wagner, G. J. (1990) Chlorosulfuron modifies biosynthesis of acyl acid substituents of sucrose esters secreted by tobacco trichomes. Plant Physiol. 94: 906-912.

Kawasaki, E. S. (1990) PCR Protocols A Guide to Methods and Applications. Edited by Innis M. A., Gelfand, D. H., Sninsky, J. J., and White, T. J. pp.21-27. Academic Press, Inc., San Diego.

Keiber, J. J., Rothenberg, M., Roman, G., Feldmann, K. A., and Ecker, J. R. (1993) CTR1, a negative regulator of the ethylene response pathway in Arabidopsis, encodes a member of the Raf family of protein kinases. Cell 72: 427-441.

Leuthy, M. H., Miernyk J. A., and Randall, D. D. (1997) Molecular analysis of a branched-chain keto-acid dehydrogenase from Arabidopsis thaliana. (abstract no. 696) Plant Physiol. 114: S-147.

Magrath, R., Bano, F., Morgner, M., Parkin, I., Sharpe, A., Lister, C., Dean, C., Lydiate, D., and Mithen, R. F. (1994) Genetics of aliphatic glucosinolates. I. Side chain elongation in Brassica napus and Arabidopsis thaliana. Heredity 72: 290-199.

Meyerowitz, E. M., and Pruitt, R. E. (1985) Arabidopsis thaliana and plant molecular genetics. Science 229: 1214-1218.

Mithen, R. F., Clarke, J., Lister, C. and Dean, C. (1995) Genetics of aliphatic glucosinolates. III. Side chain modification in Arabidopsis thaliana. Heredity 74: 210-215.

Pearson, W. R., and Lipman, D. J. (1988) Improved tools for biological sequence comparison. Proc. Natl. Acad. Sci. U.S.A. 83: 2444-2448.

Saitou, N. and Nei, M. (1987) The neighbor-joining method: a new method for reconstructing phylogenetic trees. Mol. Biol. Evol. 4(4): 406-425.

Sambrook, J., Fritsch, E. F., and Maniatis, T. (1989) Molecular Cloning A Laboratory Manual Second Edition. Cold Spring Harbor Laboratory Press, New York.

Somers, J. M., Amzallag, A., and Middleton, R. B. (1973) Genetic fine structure of the leucine operon of Escherichia coli K-12. J. Bacteriol. 113: 1268-1272.

van der Hoeven, R. S., and Steffens, J. C. (2000) Biosynthesis and elongation of short- and medium-chain-length fatty acids. Plant Physiol. 122: 275-282.

Walters, D. S., and Steffens, J. C. (1990) Branched chain amino acid metabolism in the biosynthesis of Lycopersicon pennellii glucose esters. Plant Physiol. 93: 1544-1551.

Wei, T., Maita, D., and Steffens, J. C. (1997) Cloning of two L. pennellii 2-isopropylmalate synthase cDNA and their functional expression in yeast. Direct Submission: GenBank.

Xu, H., Petersen, E I, Petersen, S B, El-Gewely, M R (1999). Random mutagenesis libraries: Optimization and simplification by PCR. BioTechniques 27:1102-1108.

U.S. Pat. No. 6,281,411

U.S. Pat. No. 6,399,861

I claim:

1. A method for enhancing the nutritional value of a plant, the method comprising:
(a) obtaining a DNA molecule with a nucleotide sequence selected from the group consisting of GenBank accession numbers AF327647, AF327648, and AY049037;
(b) mutating the DNA molecule wherein the mutated DNA encodes a protein having an isopropylmalate synthase activity with reduced feedback inhibition by L-leucine compared to a protein produced by a wild-type DNA molecule; and
(c) transforming the plant with the mutated DNA wherein the plant overproduces L-leucine compared to a non-transformed plant to enhance the nutritional value of the plant.

2. The method of claim 1, wherein the DNA molecule is AF327648.

3. A method for overproducing L-leucine in a plant, the method comprising:
(a) obtaining a DNA molecule with a nucleotide sequence selected from the group consisting of GenBank accession numbers AF327647, AF327648, and AY049037;
(b) mutating the DNA molecule;
(c) selecting the mutated DNA that encodes a protein having an isopropylmalate synthase activity with reduced feedback inhibition by L-leucine; and
(d) transforming the plant with the mutated DNA to overproduce L-leucine.

4. A method for developing a plant genetic transformation marker, the method comprising:
(a) obtaining a DNA molecule selected from the group consisting of GenBank accession numbers AF327647, AF327648, and AY049037;
(b) mutating the DNA molecule;
(c) transforming the mutated DNA into E. coli leucine auxotrophs;
(d) selecting trifluoroleucine-resistant transformed E. coli cells; and
(e) isolating the mutated DNA from the trifluoroleucine-resistant E. coli cells, said DNA molecule capable of being used as a plant genetic transformation marker.

5. An isolated DNA molecule with a nucleotide sequence selected from the group consisting of GenBank accession numbers AF327647, AF327648, and AY049037.

6. A DNA moelcule formed by mutation of a DNA molecule with a nucleotide sequence selected from the group consisting of GenBank accession number AF327647, AF327648 and AY049037, wherein the mutated DNA encodes a protein having an isopropylmalate synthase activity with reduced feedback inhibited by L-leucine compared to a protein produced by a wild type 1 non-mutatable DNA molecule.

7. A plant transformed with a mutant form of DNA, wherein the mutant form is obtained by mutating a DNA molecule of claim 5, wherein said mutant form encodes a protein having an isopropylmalate synthase activity with reduced feedback inhibition by L-leucine compared to a protein produced by a wild-type DNA molecule.

8. A vector harboring a DNA molecule of claim 6.

9. A vector harboring a mutant form of DNA, wherein the mutant form is obtained by mutating a DNA molecule of claim 5, wherein the mutant form encodes a protein having an isopropylmalate synthase activity with reduced feedback inhibition by L-leucine compared to a protein produced by a wild-type DNA molecule.

10. A cell transformed with a DNA molecule of claim 6.

11. A cell transformed with a mutant form of DNA, wherein the mutant form is obtained by mutating a DNA molecule of claim 5, wherein said mutant form encodes a protein having an isopropylmalate synthase activity with reduced feedback inhibition by L-leucine compared to a protein produced by a wild-type DNA molecule.

12. A seed transformed with a mutant form of DNA, wherein the mutant form is obtained by mutating a DNA molecule of claim 5, said mutant form encodes a protein having an isopropylmalate synthase activity with reduced feedback inhibition by L-leucine compared to a protein produced by a wild-type DNA molecule.

13. A method for producing increased levels of leucine from plans, the method comprising:
(a) obtaining a plant of claim 7; and
(b) collecting L-leucine from the plant.


SEQUENCE LISTING (45 sequences)

SEQ ID NO: 1 - protein, 631 aa, Arabidopsis thaliana
MESSILKSPNLSSPSFGVPSIPALSSSSTSPFSSLHLRSQNHRTISLTTAGKFRVSYSLSASSPLPPHAPRRRPNYIPNRISDPNYVRIFDTTLRDGEQSPGATLTSKEKLDIARQLAKLGVDIIEAGFPAASKDDFEAVKTIAETVGNTVDENGYVPVICGLSRCNKKDIETAWEAVKYAKRPRIHTFIATSDIHLKYKLKKSKEEVIEIARNMVRFARSLGCEDVEFSPEDAGRSEREYLYEILGEVIKAGATTLNIPDTVGITLPSEFGQLIADIKANTPGIQNVIISTHCQNDLGLSTANTLSGAHSGARQVEVTINGIGERAGNASLEEVVMAIKCRGDHVLGGLFTGIDTRHIVMTSKMVEEYTGMQTQPHKAIVGANAFAHESGIHQDGMLKHKGTYEIMSPEEIGLERSNDAGIVLGKLSGRHALKDRLNELGYVLDDGQLSNLFWRFKAVAEQKKRVTDADLIALVSDEVFQPEAVWKLLDMQITCGTLGLSTSTVKLADSDGKEHVACSVGTGPVDAAYKAVDLIVKEPATLLEYSMNAVTEGIDAIATTRVLIRGDNNYSSTNAVTGESVERTFSGTGAGMDIVVSSVKAYVGALNKMLGFKEHTSTLSKTPLETNEVPA

SEQ ID NO: 2 - protein, 675 aa, Arabidopsis thaliana
MASSLLRNPNLYSSTTITTTSFLPTFSSKPTPISSSFRFQPSHHRSISLRSQTLRLSCSISDPSPLPPHTPRRPRPEYIPNRISDPNYVRVFDTTLRDGEQSPGATLTSKEKLDIARQLAKLGVDIIEAGFPAASKDDFEAVKTIAETVGNTVDENGYVPVICGLSRCNKKDIERAWDAVKYAKRPRIHTFIATSDIHLEYKLKKTKAEVIEIARSMVRFARSLGCEDVEFSPEDAGRSEREYLYEILGEVIKAGATTLNIPDTVGITLPSEFGQLITDLKANTPGIENVVISTHCQNDLGLSTANTLSGAHAGARQMEVTINGIGERAGNASLEEVVMAIKCRGDHVLGGLFTGIDTRHIVMTSKMVEEYTGMQTQPHKAIVGANAFAHESGIHQDGMLKHKGTYEIICPEEIGLERSNDAGIVLGKLSGRHALKDRLTEVHSNLTHTNTCIAIFVGDTNVLLSFVHFQLGYQLDDEQLSTIFWRFKTVAEQKKRVTDADIIALVSDEVFQPEAVWKLLDIQITCGTLGLSTATVKLADADGKEHVACSIGTGPVDSAYKAVDLIVKVLFIPLRLSSTNNSPEPATLLEYSMNAVTEGIDAIATTRVLIRGSNKYSSTNAITGEEVQRTFSGTGAGMDIVVSSVKAYVGALNKMMDFKENSATKIPSQKNRVAA

SEQ ID NO: 3 - protein, 503 aa, Arabidopsis thaliana
MASLLLTSSSMITTSCRSMVLRSGLPIGSSFPSLRLTRPYDKATLFVSCCSAESKKVATSATDLKPIMERRPEYIPNKLPHKNYVRVLDTTLRDGEQSPGAALTPPQKLEIARQLAKLRVDIMEVGFPVSSEEEFEAIKTIAKTVGNEVDEETGYVPVISGIARCKKRDIEATWEALKYAKRPRVMLFTSTSEIHMKYKLKKTKEEVIEMAVNSVKYAKSLGFKDIQFGCEDGGRTEKDFICKILGESIKAGATTVGFADTVGINMPQEFGELVAYVIENTPGADDIVFAIHCHNDLGVATANTISGICAGARQVEVTINGIGERSGNAPLEEVVMALKCRGESLMDGVYTKIDSRQIMATSKMVQEHTGMYVQPHKPIVGDNCFVHESGIHQDGILKNRSTYEILSPEDVGIVKSENSGIVLGKLSGRHAVKDRLKELGYEISDEKFNDIFSRYRELTKDKKRITDADLKALVVNGAEISSEKLNSKGINDLMSSPQISAVV

SEQ ID NO: 4 - protein, 506 aa, Arabidopsis thaliana
MASSLLTSSVMIPTTGSTVVGRSVLPFQSSLHSLRLTHSYKNPALFISCCSSVSKNAATSSTDLKPVVERWPEYIPNKLPDGNYVRVFDTTLRDGEQSPGGSLTPPQKLEIARQLAKLRVDIMEVGFPGSSEEELETIKTIAKTVGNEVDEETGYVPVICAIARCKHRDIEATWEALKYAKRPRILVFTSTSDIHMKYKLKKTQEEVIEMAVSSIRFAKSLGFNDIQFGCEDGGRSDKDFLCKILGEAIKAGVTVVTIGDTVGINMPHEYGELVTYLKANTPGIDDVVVAVHCHNDLGLATANSIAGIRAGARQVEVTINGIGERSGNASLEEVVMALKCRGAYVINGVYTKIDTRQIMATSKMVQEYTGLYVQAHKPIVGANCFVHESGIHQDGILKNRSTYEILSPEDIGIVKSQNSGLVLGKLSGRHAVKDRLKELGYELDDEKLNAVFSLFRDLTKNKKRITDADLKALVTSSDEISLEKLNGANGLKSNGYIPVPQVSSNV

SEQ ID NO: 5 - protein, 589 aa, Lycopersicon pennellii
MFFFLQLLVPIISVFQSKKHYYSTFIRCSISNRRPEYVPSKISDPKYVRIFDTTLRDGEQSPGATMTTKEKLDVARQLAKLGVDIIEAGFPASSEADFESVKLIAEEIGNNTDENGFVPVICGLSRCNKSDIDKAWEAVKYAKKPRVHTFIATSEIHMKYKLKMSREQVVEKARSMVAYARSLGCEDVEFSPEDAGRSDREFLYDILGEVIKAGATTLNIPDTVGYTVPSEFGQLITDIKANTPGIENVIISTHCQNDLGLSTANTLAGACAGARQLEVTINGIGERAGNASLEEVVMALKCRGEQVLGGLYTGINTQHIVPSSKMVEEYSGLQVQPHKAIVGANAFAHESGIHQDGMLKHKDTYEIISPDDVGLSRSNEAGIVLGKLSGRHALKSKMLELGYDIDGKELEDLFWRFKSVAEKKKKITDDDLIALMSDEVLQPNVYWKLGDVQIMCGSLGLSTATVKLINTDGQEHIACSVGTGPVDAAYKAVDLIVKVPITLLEYSMNAVTEGIDAIASTRVSICSIDRHTIMNGSTGQTIHRTFSGTGADMDVVISSVRAYIGALNKMLSYEKLVSRYSKPEDSVVV

SEQ ID NO: 6 - protein, 612 aa, Lycopersicon pennellii
MASITANHPISGKPLISFRPKNPLLQTQTLFNFKPSISKHSNSSFSIPVVRCSIRRIPEYTPSHIPDPNYVRIFDTTLRDGEQSPGATMTTKEKLDVARQSAKLGVDIIEAGFPASSEADLEAVKLIAKEVGNGVYEEEYVPVICGLARCNKKDIDKAWEAVKYAKKPRIHTFIATSEVHMNYKLKMSRDQVVEKARSMVAYARSIGCEDVEFSPEDAGRSDPEFLYHILGEVIKAGATTLNIPDTVGYTVPEEFGQLIAKIKANTPGVEDVIISTHCQNDLGLSTANTLAGACAGARQLEVTINGIGERAGNASLEEVVMALKCRGEQVLGGLYTGINTQHILMSSKMVEGISGLHVQPHKAIVGANAFVHESGIHQDGMLKHKDTYEIISPEDIGLNRANESGIVFGKLSGVMLCKPKMLELGYEIEGKELDDLFWRFKSVAEKKKKITDDDLVALMSDEVFQPQFVWQLQNVQVTCGSLGLSTATVKLIDADGREHISCSVGTGPVDAAYKAVDLIVKVPVTLLEYSMNAVTQGIDAIASTRVLIRGENGHTSTHALTGETVHRTFSGTGADMDIVISSVRAYVGALNKMMSFRKLMAKNNKPESSAVI

SEQ ID NO: 7 - protein, 565 aa, Glycine max
MPTKTSTPSSQSPKLSHLRPQYIPNHIPDSSYVRILDTTLRDGEQSPGATMTAKEKLDIARQLVKLGVDIIQPGFPSASNSDFMAVKMIAQEVGNAVDDDGYVPVIAGFCRCVEKDISTAWEAVKYAKRPRLCTSIATSPIHMEHKLRKSKDQVIQIARDMVKFARSLGCNDIQFGAEDATRSDREFLYEILGVVIEAGATTVNIADTVGIVMPLELGKLIVDIKDNTPGIANVIISTHCHNDLGLATANTIEGARTGARQLEVTINGIGERAGNASLEEVVMALASKGDHALNGLYTRINTRHILETSKMVEEYSGMHLQPHKPLVGANAFVHASGIHQDGMLKHKGTYETISPEEIGHKRTTRIGIVLGKLSGSQALRKRLEELGYDLKEDEVDSVFWQFKAMAEKKKVVTDVDLKALVSYKAFHAESIWKLGDLQVTCGTIGLSTATVKLVNIDGSTHVACSIGIGAVDSTYKAINLIVKEPTKLLDYSLNSVTEGIGVNVTARVVICRENNHTSTYAFTEDANYPTFSGIAAEMDVVVSTVKAYLVALNKLLRWKESFRCA

SEQ ID NO: 8 - protein, 584 aa, Schizosaccharomyces pombe
MKSTFEAAGRVAKGMLKDPSKKYKPFKGIQLPNRQWPNKVLTKAPRWLSTDLRDGNQALPDPMNGQEKLRYFKLLCSIGFKEIEVGFPSASQTDFAFVRHLIETPGLIPDDVTISALTPSREPLILRTIEALRGAKNATVHLYNACSPLFREVVFRNSKQETLDLAIKGSKIVTAATKNALESKETNWGFEYSPETFSDTEPDFALEVCEAVKGMWKPSAAQPIIFNLPATVEMSTPNTYADLIEYFSTNISEREKVCVSLHPHNDRGTAVAAAELGQLAGGDRIEGCLFGNGERTGNVDLVTLAFNLYTQGVSPNLDFSKLDEIIRITEDCNKINVHPRHPYAGNLVFTAFSGSHQDAISKGLKAYDERKAVDPVWKVPYLPLDPHDVNSEYAAIIRVNSQSGKGGVAYLLKTNCGLDLPRALQVEFGSIVKDYSDTKGKELSIGEISDLFYTTYYLEFPGRFSVNDYTLSSNGPQSKCIKCVVDIKGEKKDTPSRVVIEGVGNGPLSALVDALRRQFNISFDIGQYSEHAIGSGNGVKAASYVEIIFNNTSFWGVGIDADVTSAGLKAVMSGVSRASRAFAK

SEQ ID NO: 9 - protein, 619 aa, Saccharomyces cerevisiae
MVKESIIALAEHAASRASRVIPPVKLAYKNMLKDPSSKYKPFNAPKLSNRKWPDNRITRAPRWLSTDLRDGNQSLPDPMSVEQKKEYFHKLVNIGFKEIEVSFPSASQTDFDFTRYAVENAPDDVSIQCLVQSREHLIKRTVEALTGAKKATIHTYLATSDMFREIVFNMSREEAISKAVEATKLVRKLTKDDPSQQATRWSYEFSPECFSDTPGEFAVEICEAVKKAWEPTEENPIIFNLPATVEVASPNVYADQIEYFATHITEREKVCISTHCHNDRGCGVAATELGMLAGADRVEGCLFGNGERTGNVDLVTVAMNMYTQGVSPNLDFSDLTSVLDVVERCNKIPVSQRAPYGGDLVVCAFSGSHQDAIKKGFNLQNKKRAQGETQWRIPYLPLDPKDIGRDYEAVIRVNSQSGKGGAAWVILRSLGLDLPRNMQIEFSSAVQDHADSLGRELKSDEISKLFKEAYNYNDEQYQAISLVNYNVEKFGTERRVFTGQVKVGDQIVDIEGTGNGPISSLVDALSNLLNVRFAVANYTEHSLGSGSSTQAASYIHLSYRRNADNEKAYKWGVGVSEDVGDSSVRAIFATINNIIHSGDVSIPSLAEVEGKNAAASGSA

SEQ ID NO: 10 - protein, 518 aa, Buchnera aphidicola
MNSQVIIFDTTLRDGEQALQASLSVKQKLQIALSLENAGIDIIEVGFPISSPGDFKSVQTISKNIKNSRICSLARCLNKDIDTAAEAMSSSNTFRIHIFLATSTLHMESKLKKNFDQIIDMAISSVKRALRYTDDVEFSCEDASRTTMDNLCRIVEQLIKAGVKTINIPDTVGYTVPNELSTIIHNLFKRVPNIDQSIISVHCHNDLGMAVGNSISAIQAGARQIEGTINGIGERAGNTALEEVIMAIKVREDILGVSTNIKHKEIYRTSQIISQICNLPIPPNKAIVGSNAFAHSSGIHQDGVLKNRKNYEIMEPSSIGLKEVKLNLTSRSGRAAVKYYMTQMGYKECDYNIDELYTSFLKLADKKGQVFDYDLEALAFINMQQEESEYFSLSFFSVQSISNGLSTASVKLLCGKKVSIESATTSNGPIDAIYQALNRITNFPITLQKYQLVAKGKGRDALGQVDILVEYKKRKFHGVGLATDIIESSAQAMVNVLNNIWKANQVNEKLKTLKKVNN

SEQ ID NO: 11 - protein, 523 aa, Escherichia coli
MSQQVIIFDTTLRDGEQALQASLSVKEKLQIALALERMGVDVMEVGFPVSSPGDFESVQTIARQVKNSRVCALARCVEKDIDVAAESLKVAEAFRIHTFIATSPMHIATKLRSTLDEVIERAIYMVKRARNYTDDVEFSCEDAGRTPIADLARVVEAAINAGATTINIPDTVGYTMPFEFAGIISGLYERVPNIDKAIISVHTHDDLGLAVGNSLAAVHAGARQVEGAMNGIGERAGNCSLEEVIMAIKVRKDILNVHTAINHQEIWRTSQLVSQICNMPIPANKAIVGSGAFAHSSGIHQDGVLKNRENYEIMTPESIGLNQIQLNLTSRSGRAAVKHRMDEMGYKESEYNLDNLYDAFLKLADKKGQVFDYDLEALAFIGKQQEEPEHFRLDYFSVQSGSNDIATAAVKLACGEEVKAEAANGNGPVDAVYQAINRITEYNVELVKYSLTAKGHGKDALGQVDIVANYNGRRFHGVGLATDIVESSAKAMVHVLNNIWRAAEVEKELQRKAQHNENNKETV

SEQ ID NO: 12 - protein, 515 aa, Haemophilus influenzae
MTDRVIIFDTTLRDGEQALKASLTVKEKLQIALALERLGVDVMEVGFPVSSQGDFESVQTIARHIKNARVAALSRAVDKDIDAAYEALKVAEAFRIHTFIASSALHVEAKLKRSFDDVVGMAVAAVKRARNYTDDVEFSCEDAGRTGIDNICRIVEAAINAGATTVNIPDTVGFCLPNEYGNIIAQVRNCVPNIDKAVISVHCHNDLGMATANSLTAVQNGARQIECTINGIGERAGNTSLEEVVMAMKVRQDFMGVDTHINTQEIHRVSQMVSQLCNMPIQPNKAIVGSNAFAHSSGIHQDGMLKNKNTYEILSPETIGLKKEKLNLTARSGRAAVKGHMADMGYNEQDYDLDKLYDEFLKLADKKGQVFDYDLEALAFIDMQQGDEDRLVLDKLSAHSTKEYPATAFVQLKLDGEKLSTSSIGGNGPVDAVYNAILNLTGLEIKMSHYNLTAKGEGAEALGQVDIVVEHKGRKFHGVGLATDIVESSALALVHAINAIYRAHKVADIKNHKHH

SEQ ID NO: 13 - protein, 513 aa, Lactococcus lactis
MRKIEFFDTSLRDGEQTPGVSFSISEKVTIAKQLEKWRISVIEAGFSAASPDSFEAVKQIADSLNDTAVTALARCVISDIDKAVEAVKGAKYPQIHVFIATSPIHMKYKLKISPEEVLKNIDKCVRYARERVEVVEFSPEDATRTELNFLLEAVQTAVDAGATYINIPDTVGYTTPEEYGKIFKFLIDNTKSDREIIFSPHCHDDLGMAVANSLAAIKAGAGRVEGTVNGIGERAGNAALEEIAVALHIRKDFYQAQSPLKLSETAATAELISQFSGIAIPKNKAIVGANAFAHESGIHQDGVLKNAETYEIITPELVGIKHNSLPLGKLSGRHAFSEKLTELNIAYDDESLAILFEKFKKLADKKKEITDADIHALFTGETVKNLAGFILDNVQIDGHKALVQLKNQEEEIYVSQGEGSGSVDAIFKAIDKVFNHQLKLISYSVDAVTDGIDAQATTLVSVENLSTGTIFNAKGVDYDVLKGSAIAYMNANVLVQKENLQGKVEQISAHDGI

SEQ ID NO: 14 - protein, 533 aa, Microcystis aeruginosa
MNTSPDRVIIFDTTLRDGEQSPGAALNVDEKLTIARALARLGVDVIEAGFPHASPGDFEAVQKIAGSVGSEADSPIICGLARATQKDIKSAADALRPAAKPRIHTFLATSDIHLQYKLKKTRQEVLEIVPEMVAYAKSFLNDVEFSPEDAGRSDPEFLYQVLERAIAAGATTVNIPDTVGYTTPSEFGALIRGIKENVPNIDQAIISVHGHDDLGLAVANFLEAVKNGARQLECTINGIGERAGNASLEELVMALHVRRSYFNPFLGRPAESTEPLTKINTKEIYRTSRLVSNLTGMIVQPNKAIVGANAFAHESGIHQDGVLKHKLTYEIMDAESIGLTNNQIVLGKLSGRNAFRSRLQELGFELSETELNNAFIQFKEMADRKKEITDRDLEAIVNDEIDTVPDHFRLELVQVSCGNNARPTATVTIRTPDGSELSDAAIGTGPVDALCKAIDRVVQIPNELISFSVREVTEGIDALGEVTIRLRYAGRTYSARAADTDIIVASARAYVSALNRLHVALQQKEKTPEMLQV

SEQ ID NO: 15 - protein, 573 aa, Streptomyces coelicolor
MANRQQPSPMPTAKYRGYDQVDIADRTWPNQRITTAPRWLSTDLRDGNQALIDPMSPVRKRAMFDLLVKMGYKVIEVGFPASGQTDFDFVRSIIEEPGAIPDDVTISVLTQAREDLIERTVESLKGARRATVHLFNATAPVFRRVVFRGSRDDIKQIAVDGTRLVMEYAEKLLGPETEFGYQYSPEIFTDTELDFALEVCEAVMDTYQPGPGREIILNLPATVERSTPSTHADRFEWMGRNLSRREHVCLSVHPHNDRGTAVAAAELALMAGADRIEGCLFGQGERTGNVDLVTLGMNLFSQGVDPQIDFSDIDEIRRTWEYCNQMEVHPRHPYVGDLVYTSFSGSHQDAIKKGFDAMEADAAARGVTVDDIEWAVPYLPIDPKDVGRSYEAVIRVNSQSGKGGIAYVLKNDHSLDLPRRMQIEFSKLIQAKTDAEGGEITPTAIWDVFQDEYLPNPDNPWGRIQVANGQTTTDRDGVDTLTVDATVDGAETTLVGSGNGPISAFFHALQGVGIDVRLLDYQEHTMSEGASAQAASYIECAIGDKVLWGIGIDANTTRASLKAVVSAVNRATR

SEQ ID NO: 16 - protein, 533 aa, Synechococcus PCC6803
MNSPVDRILIFDTTLRDGEQSPGATLTVEEKLSIARALARLGVDIIEAGFPFASPGDFEAVQKIAQTVGTENGPVICGLARATQKDIKAAAEALKPAAKHRIHTFLATSDIHLEHKLKKTRAEVLAIVPEMVAYAKSLVNDIEFSPEDAGRSDPEFLYQVLEAAISAGATTINIPDTVGYTTPAEYGALIKGIADNVPNIDQAIISVHGHNDLGLAVANFLEAVKNGARQLECTINGIGERAGNAALEELVMALHVRRSYFNPFLGRPADSTAPLTNIDTKHIYATSRLVSELTGMMVQPNKAIVGANAFAHESGIHQDGVLKNKLTYEIMDAESIGLTNNQIVLGKLSGRNAFGTRLKELGFDLSDTELNNAFIRFKEVADKRKEITDWDLEAIVNDEIRQPPELFRLERVQVSCGEPSVPTATLTIRTPAGPEETAVAIGTGPVDAVYKAINQIVQLPNELLEYSVTSVTEGIDALGKVSVRLRHNGVIYTGYAANTDIIVASARAYLGALNRLYAALEKSREHPPVVASL

SEQ ID NO: 17 - DNA, 214 bp, Arabidopsis thaliana
accgatgctgacttaatagctttagtatctgatgaagtgtttcagccagaagctgtctggaaactcctggacatgcagataacttgtggaactctcggtctctcaacatctactgtaaaacttgctgactccgatggcaaagagcatgtagcttgttctgttggaaccggacctgtagatgcagcttacaaggcagttgatcttatcgttaagg

SEQ ID NO: 18 - DNA, 201 bp, Arabidopsis thaliana
actgatgcggacataatagctttagtatctgatgaagttttccagccagaagaactgtaaaacttgctgacgctgatggcaaagaacatgtcgcttgttctattggaactgggcctgtggattcagcttacaaggcagtagatcttatcgtaaaggtactgtttataccgctgaggctctcatctacaaataactcaccag

SEQ ID NO: 19 - DNA, 176 bp, Arabidopsis thaliana
tcagtgatgagaaattcaacgacatcttctcacgatacagagaattaacgaaggacaaaaagagaatcacagacgctgatctgaaggcattagtggtgaacggtgctgaaatctcatcagaaaaattaaacagtaaaggaattaacgaccttatgtcaagccctcagatttccgct

SEQ ID NO: 20 - DNA, 185 bp, Arabidopsis thaliana
tcgatgatgagaaattgaacgctgtcttctcactattcagagatttaaccaagaataaaaagagaatcacggatgctgatttgaaggcattagtaacatctagcgatgaaatctctttggagaaattaaacggcgctaacggtttaaagtctaacggctatataccagttcctcaggtttcatcg

SEQ ID NO: 21 - protein, 8 aa, Artificial Sequence
  Description of Artificial Sequence Illustrative conserved peptide
TTLRDGEQ

SEQ ID NO: 22 - protein, 9 aa, Artificial Sequence
  Description of Artificial Sequence Illustrative conserved peptide
NGIGERAGN

SEQ ID NO: 23 - protein, 7 aa, Artificial Sequence
  Description of Artificial Sequence Illustrative conserved peptide
SGIHQDG

SEQ ID NO: 24 - protein, 17 aa, Artificial Sequence
  Description of Artificial Sequence Illustrative consensus sequence
LRXGXQXXXXXXXXXXK

SEQ ID NO: 25 - protein, 14 aa, Artificial Sequence
  Description of Artificial Sequence Illustrative consensus sequence
XXXHXHXDXGXXXX

SEQ ID NO: 26 - DNA, 21 bp, Artificial Sequence
  Description of Artificial Sequence Primer
ccacacctatctcctcctctt

SEQ ID NO: 27 - DNA, 21 bp, Artificial Sequence
  Description of Artificial Sequence Primer
cctgcatcttctggactgaac

SEQ ID NO: 28 - DNA, 21 bp, Artificial Sequence
  Description of Artificial Sequence Primer
gtggttggccggtcagtgtta

SEQ ID NO: 29 - DNA, 21 bp, Artificial Sequence
  Description of Artificial Sequence Primer
cacagtcttggcgatggtctt

SEQ ID NO: 30 - DNA, 28 bp, Artificial Sequence
  Description of Artificial Sequence Primer
caggtaccatggcttcatcgcttctgac

SEQ ID NO: 31 - DNA, 29 bp, Artificial Sequence
  Description of Artificial Sequence Primer
gggagctcttacacattcgatgaaacctg

SEQ ID NO: 32 - DNA, 20 bp, Artificial Sequence
  Description of Artificial Sequence Primer
ttatctgcatgtccaggagt

SEQ ID NO: 33 - DNA, 20 bp, Artificial Sequence
  Description of Artificial Sequence Primer
catcagagattctcctcgac

SEQ ID NO: 34 - DNA, 20 bp, Artificial Sequence
  Description of Artificial Sequence Primer
cgaattcttcctcagacgac

SEQ ID NO: 35 - DNA, 24 bp, Artificial Sequence
  Description of Artificial Sequence Primer
gaggccaaggatactcgtattcac

SEQ ID NO: 36 - DNA, 24 bp, Artificial Sequence
  Description of Artificial Sequence Primer
ccatggtattagacacgacgcttc

SEQ ID NO: 37 - DNA, 26 bp, Artificial Sequence
  Description of Artificial Sequence Primer
tctagacggccgctttattcattaca

SEQ ID NO: 38 - DNA, 26 bp, Artificial Sequence
  Description of Artificial Sequence Primer
ccatggagtcttcgattctcaaaagc

SEQ ID NO: 39 - DNA, 26 bp, Artificial Sequence
  Description of Artificial Sequence Primer
tctagagattttcttcaggcagggac

SEQ ID NO: 40 - DNA, 28 bp, Artificial Sequence
  Description of Artificial Sequence Primer
gctctagaactgatgcggacataatagc

SEQ ID NO: 41 - DNA, 28 bp, Artificial Sequence
  Description of Artificial Sequence Primer
gcggtaccctggtgagttatttgtagat

SEQ ID NO: 42 - DNA, 28 bp, Artificial Sequence
  Description of Artificial Sequence Primer
gcgagctccgatgatgagaaattgaacg

SEQ ID NO: 43 - DNA, 28 bp, Artificial Sequence
  Description of Artificial Sequence Primer
gactcgagcgatgaaacctgaggaactg

SEQ ID NO: 44 - DNA, 26 bp, Artificial Sequence
  Description of Artificial Sequence Primer
gcctcgagctgatgagaacgaagatg

SEQ ID NO: 45 - DNA, 26 bp, Artificial Sequence
  Description of Artificial Sequence Primer
ccggtacctatctgagtcgcttctgc